(19) United States
(12) Patent Application Publication
(10) Pub. No.: US 2025/0009401 A1
(43) Pub. Date: Jan. 9, 2025

(54) BONE FIXATION DEVICE FOR BONE FIXATION

(71) Applicant: Ioannis VLAMIS, Ekali (GR)

(72) Inventor: Ioannis VLAMIS, Ekali (GR)

(21) Appl. No.: 18/765,254

(22) Filed: Jul. 6, 2024

Related U.S. Application Data
(60) Provisional application No. 63/525,305, filed on Jul. 6, 2023.

(51) Int. Cl. A61B 17/82; A61B 17/16; A61B 17/74

(52) U.S. Cl. CPC A61B 17/823; A61B 17/1697; A61B 17/74

(57) ABSTRACT

A bone fixation device for fixation of a first bone and a second bone to each other in a bone fracture and/or a joint dislocation includes a first fixation element, a second fixation element and a flexible strand. The first fixation element is configured for passing through a hole of the first bone and a hole of the second bone and for engaging the second bone. The second fixation element is configured for engaging the first bone and for securing the flexible strand. The first fixation element is configured for receiving the flexible strand and includes a hollow cylinder, wherein the hollow cylinder extends in a direction of a longitudinal axis from a first end to a second end and has a wall defining a cylinder hollow space.

### CROSS REFERENCE TO RELATED APPLICATIONS

The present application claims priority to U.S. Provisional Application No. 63/525,305 filed on Jul. 6, 2023, the entire teachings of which are hereby incorporated by reference.

### BACKGROUND OF THE INVENTION

### Field of the Invention

The invention relates to a bone fixation device for fixation of a first bone and a second bone to each other in a bone fracture and/or a joint dislocation, and in particular, to a bone fixation device for bone fixation in a lesser trochanter fracture, in which at least part of the lesser trochanter is detached from the femur bone resulting in a lesser trochanteric fragment, or for bone fixation in an acromioclavicular joint dislocation.

### Description of the Related Art

The lesser trochanter fracture is a type of hip fracture that may occur as an isolated fracture or as a component of another type of hip fracture. In the latter case, the other type of fracture is the main fracture and the lesser trochanter fracture the secondary fracture. The main fracture can in particular be an intertrochanteric fracture, which is an extracapsular fracture of the proximal femur that occurs between the greater trochanter and the lesser trochanter. The greater trochanter serves as an insertion site for the gluteus medius, the gluteus minimus, the obturator internus, and the piriformis as well as the site of origin for the vastus lateralis muscle, while the lesser trochanter serves as an insertion site for the iliopsoas muscle.

Both as an isolated fracture and as a secondary fracture, the lesser trochanteric fragment needs to be reduced, i.e. to be brought to the right position close the femur bone, and fixed to the femur bone so that healing can occur. In particular, in the case of an intertrochanteric fracture as the main fracture, the parameters of the lesser trochanter fracture such as the displacement of the lesser trochanteric fragment, and the extent to which the lesser trochanteric fragment is stabilized may affect the treatment of the intertrochanteric fracture. Thus, reduction and fixation of the lesser trochanter is necessary to achieve optimal results also in the treatment of the intertrochanteric fracture.

Currently, reduction and fixation of the lesser trochanteric fragment in a lesser trochanter fracture is done by screws or metal wire. This way of treatment requires an extensive surgical approach that increases surgical time and blood loss, and has a higher risk of postoperative complications. Further, surgical options for treating an intertrochanteric fracture include sliding hip screws and intramedullary nails. In particular, for intertrochanteric fractures with a lesser trochanter fracture as a secondary fracture, intramedullary nailing is the gold standard option. However, the reduction and stabilization of the lesser trochanter fracture is not possible with the existing nails. The instability caused by the fracture of the lesser trochanter can in turn cause cut-out and failure of fixation of the intertrochanteric fracture.

The acromioclavicular joint (ACJ) is one of the more common sites of shoulder girdle injury, accounting for 4% to 12% of all such injuries, with an incidence of 3 to 4 cases per 100 000 persons per year in the general population and is more common in males and athletes.

An acromioclavicular joint dislocation (ACJ dislocation) can lead to instability and cause persistent pain and functional impairment. It is often sustained while falling on to the shoulder and the dislocation is classified according to the distance and direction of the clavicle from the acromion. According to Rockwood, ACJ dislocation can be classified in six types (I to VI).

Mechanical trauma of the ACJ can tear the ligamentous apparatus that holds the acromion, clavicle, and coracoid process together. Persistent instability can lead to chronic, painful limitation of shoulder function, particularly with respect to working above the head.

Surgical stabilization is therefore recommended for high-grade instability of Rockwood types III, IV and V. The preferred surgical treatment is open reduction and internal fixation (ORIF). This can be done by suture, hook plate, coracoclavicular screw, cortical button and K-wire stabilization.

Modern reconstruction techniques with cortical button enable selective vertical and horizontal treatment of the instability and have been found superior to traditional methods, particularly in young athletes. Still the surgical technique has a degree of complexity and danger. It requires an extensive approach to the coracoid process and the advancement of the button below the coracoid is sometimes difficult and may result in longer operative time and possible damage to neurovascular structures.

### BRIEF SUMMARY OF THE INVENTION

It is an object of the present invention to provide a bone fixation device for fixation of a first bone and a second bone to each other in a bone fracture and/or a joint dislocation in a minimally invasive, precise and stable way. Another object of the present disclosure is to provide a bone fixation method for fixation of a first bone and a second bone to each other in a bone fracture and/or a joint dislocation by means of a bone fixation device.

The bone fixation device for fixation of a first bone and a second bone to each other in a bone fracture and/or a joint dislocation includes a first fixation element and a flexible strand. The first fixation element is configured/adapted for passing through a hole of a first bone and a hole of a second bone and for engaging the second bone. The first fixation element includes a hollow cylinder. In particular, the first fixation may be formed as a hollow cylinder. The hollow cylinder extends in a direction of a longitudinal axis from a first end to a second end and has a wall that defines a cylinder hollow space. The first fixation element is configured/adapted for receiving the flexible strand. Within the context of the present invention, a bone can in particular be a whole bone or part of a bone such as a bone fragment. The first end and the second end of the hollow cylinder may preferably correspond to a first end and a second end of the first fixation element.

With the bone fixation device of the present invention, the first bone and the second bone can be fixed to each other in a minimally invasive, stable, precise and accurate way. The bone fixation device does not require an extensive surgical approach but only gaining access to the first bone and forming a hole in the first bone and a hole in the second bone for the first fixation element to pass through. Due to the minimally invasive character of the bone fixation that can be achieved by using the bone fixation device of the present invention, the operation time and the risk of intraoperative and postoperative complications can be reduced. Due to the simple design of the first fixation element, in particular if the cylindrical shape of the outer circumferential surface of the first fixation element matches a cylindrical shape of the hole of the first bone and the hole of the second bone, the surgeon can intuitively pass the first fixation element through the hole of the first bone and the hole of the second bone and thus easily perform the reduction and fixation of the first bone and the second bone to each other. Moreover, the hollow design of the first fixation element enables the first fixation element to be used together with a guiding element, in particular a guidewire, over which the first fixation element can be guided through the hole of the first bone and the hole of the second bone. The hollow cylinder is advantageously formed such that the cylinder hollow space can receive the guiding element or in other words such that the guiding element can be passed through the cylinder hollow space of the first fixation element. Thus, the preferred position of the first fixation element with respect to the second bone can be easily and accurately achieved, while the position of the first fixation element relative to the hole of the first bone and the hole of the second bone can be controlled during the whole procedure. A cross-section of the guiding element may preferably have a maximum linear dimension, in particular a maximum diameter, that is larger than 2 mm and smaller than 3.5 mm. A further advantage of the bone fixation device of the present invention lies in that, due to its design, the first fixation element can easily be passed through the hole of the first bone and the hole of the second bone by being pushed through the holes.

When the bone fixation device is used for reduction and bone fixation in a bone fracture, the bone fixation device has the advantage of healing of the bone fracture with reduced pain. These effects can advantageously be achieved due to the aforementioned design of the bone fixation device of the present invention that enables a bone-to-bone attachment between the first bone and the second bone. Here, the second bone may in particular be a bone fragment. In this case, the first bone corresponds to a bone from which the bone fragment corresponding to the second bone is detached. The term “bone-to-bone attachment” means that the first bone and the second bone are directly attached to each other without intervening means such as a screw that would otherwise engage both the first bone and the second bone at the fracture line. In the present invention, the second bone (in particular, corresponding to the bone fragment) in a bone fracture is advantageously fixed to the first bone by pressing the second bone against the first bone by the first fixation element. To this end, the first fixation element can be pulled, thereby engaging the second bone. This can be achieved by pulling the flexible strand received by the first fixation element. The flexible strand may then for example be attached, in particular tied, to a bone bridge formed on a proximal side of the first bone. A bone bridge is in particular a region of bone that connects two areas of bone separated from each other e.g. by holes formed in the bone. If the bone fixation device includes a second fixation element according to an advantageous embodiment, as will be described later, fixing the second bone to the first bone can advantageously be achieved by the flexible strand pulling the first fixation element and the second fixation element towards each other. Thus, by using the bone fixation device of the present invention, bone tissue (i.e. bone as a material) at the fracture line can more easily grow and heal so that the second bone is eventually naturally fixed to the first bone.

When the bone fixation device is used for reduction of a joint, in particular of the second bone, in a joint dislocation, the treatment of the joint dislocation can be easily performed by the surgeon, while reducing at the same time the operation duration and the risk of intraoperative and postoperative complications. It is noted that bone fixation in a joint dislocation includes bringing the first bone and the second bone to a new position relative to each other, in particular the initial position before the occurrence of the joint dislocation, and fixing them to each other in this position so that the joint dislocation is fixed and its symptoms are alleviated. The new position may be characterized as an end position, while the position when the joint is dislocated may be characterized as a start position. The distance between the first bone and the second bone at the start position is in particular larger than the distance between the first bone and the second bone at the end position. Further, in the case of a ligament tear, the ligaments of the joint can be healed. The first bone and the second bone may be bones that are themselves part of the joint or that can be manipulated and fixed to each other for treating the joint dislocation.

Within the context of the present invention, a first fixation element that is formed as a hollow cylinder, i.e. having the shape of a hollow cylinder, can also be described as hollow-cylindrically shaped. Here, features of the first fixation element can also be referred to as features of the hollow cylinder or of the hollow-cylindrically shaped first fixation element.

The cylinder hollow space advantageously extends from the first end to the second end of the hollow cylinder.

It is understood that the hollow cylinder advantageously has, in addition to the cylinder hollow space, an outer circumferential surface, an inner circumferential surface, and two bases. The cylinder hollow space is advantageously surrounded/enclosed by the inner circumferential surface. The outer circumferential surface and the inner circumferential surface can advantageously have the same or different (cylindrical) shapes, in particular the same or different contours or shapes of curves produced by the intersection of the surfaces with a normal plane (in other words the same or different normal sections). A normal plane is any plane containing the normal vector of a corresponding surface at a particular point. For example, both the outer circumferential surface and the inner circumferential surface may be circular cylindrical surfaces or in other words have circular normal sections. In this example, the hollow cylinder has an annular cross-section. In an alternative example, the outer circumferential surface may have a circular normal section and the inner circumferential surface an elliptical normal section or vice versa. In this alternative example, the hollow cylinder has a cross-section with a circular outer contour and an elliptical inner contour or vice versa.

The hole of the first bone and the hole of the second bone can advantageously be considered to form a bone hole. The hole of the first bone and the hole of the second bone are advantageously formed such that they are aligned when the second bone is at the right position relative to the first bone for the fixation of the first bone and the second bone to each other to take place as well as when the second bone is reduced and fixed to the first bone. In the fixed state of the second bone in the case of using the bone fixation device in a bone fracture, the hole of the first bone and the hole of the second bone advantageously form a continuous bone hole. It is understood that the hole of the first bone is a hole through the first bone and that the hole of the second bone is a hole through the second bone.

Preferably, the hole of the first bone and/or the hole of the second bone has/have a circular cross-section.

Preferably, the hole of the first bone and/or hole of the second bone has/have a diameter between 3.7 mm and 4.3 mm, more preferably between 3.9 mm and 4.1 mm, in particular 4 mm.

For achieving the reduction and fixation of the first bone and the second bone to each other, the first fixation element is advantageously passed, in particular pushed, through the hole of the first bone and the hole of the second bone so that it exits/comes out of the second bone (second bone hole)/until it has exited/come out of the second bone (second bone hole), with the flexible strand received by/from/in the first fixation element. The first fixation element is advantageously passed through the hole of the first bone and the hole of the second bone while a first end region of the flexible strand and a second end region of the flexible strand are located outside the hole of the first bone, in particular held the surgeon.

When the first fixation element exits/comes out of the second bone, the first fixation element falls/rotates/pivots or, seen from the side of the surgeon, is let to fall/rotate/pivot, so that the first fixation element comes into a position for engaging the second bone, in particular its distal side. To this end, the surgeon may advantageously reduce the tension of/release the flexible strand as soon as the first fixation element is out of the hole of the second bone. Then, the surgeon can again increase the tension of/pull the flexible strand so that the first fixation engages the second bone such that, in the case of a bone fracture, the first bone and the second contact each other, in particular are pressed against each other, and in the case of a joint dislocation, brought closed to each other, in particular pulled to each other.

Preferably, the hollow cylinder has an inner circumferential surface that has a circular cross-section.

Preferably, the hollow cylinder is a right hollow cylinder.

According to a preferred embodiment of the present invention, the hollow cylinder is a right circular hollow cylinder. The right circular hollow cylinder has an annular cross-section. This means that both the inner circumferential surface and the outer circumferential surface have circular normal sections. A right circular hollow cylinder can in particular be understood as a three-dimensional region bounded by two right circular cylinders having the same axis and two parallel annular bases perpendicular to the cylinders' common axis. In particular, the cross-section of the first fixation element may preferably be of annular shape.

The flexible strand is preferably a suture strand. However, any type of thread that is suitable for surgical operations can be used as the flexible strand of the bone fixation device. The flexible strand is advantageously chosen such it has the necessary strength to fix the first bone and the second bone to each other. If a second fixation element is provided, the flexible strand is advantageously used to connect the first fixation element to the second fixation element.

The flexible strand may advantageously have a length that is larger than twice the distance between a proximal bone surface (proximal cortex) of the first bone and the distal bone surface (distal cortex) of the second bone so that it can be sufficiently long for the first fixation element to exit hole of the second bone. The distance is advantageously a distance in the direction of a longitudinal axis of the bone hole through which the first fixation element is intended to pass through, in particular the minimum distance between the proximal cortex of the first bone and the distal cortex of the second bone.

Preferably, an outer diameter of the hollow cylinder is smaller than or equal to 4 mm. Preferably, the outer diameter is larger than or equal to 3 mm. In particular, it may preferably lie between 3 mm and 4 mm. The outer diameter of the hollow cylinder is advantageously smaller than the diameter of the hole of the first bone and the diameter of the hole of the second bone

The hollow cylinder has advantageously a length that is larger than a diameter of the hole of the second bone. Preferably, the length of the hollow cylinder is larger than or equal to 12 mm and/or smaller than or equal to 17 mm. In particular, it lies between 12 mm and 17 mm. Thus, the first fixation element has a suitable length for stably engaging the second bone while being compact at the same time.

Preferably, a thickness of the wall of the hollow cylinder lies between 0.5 mm and 1 mm. Due to the chosen thickness of the wall, the first fixation element can be compact. Further, the cylinder hollow space can be provided with such a dimension, in particular diameter, that enables the first fixation element to be easily guided over a guiding element, in particular guidewire.

According to an advantageous embodiment, the cylinder hollow space is configured for receiving the flexible strand. This means in particular that the cylinder hollow space is configured such that the flexible strand can be placed inside and/or passed through the cylinder hollow space. In particular, the cylinder hollow space can be used for passing the flexible strand through it, wherein the first fixation element can then be passed through the hole of the first bone and the hole of the second bone.

Preferably, the first fixation element further includes at least one opening that is configured for receiving the flexible strand. Within the context of the present invention, the expression that the at least one opening of the first fixation element is configured for receiving the flexible strand means in particular that the at least one opening is configured such that the flexible strand can be placed inside and/or passed through the at least one opening. The at least one opening may include one or a plurality of openings. The at least one opening may preferably include an opening formed in and/or on the wall of the hollow cylinder.

According to an advantageous embodiment, the at least one opening may include a wall hole formed in the wall of the hollow cylinder. In particular, the wall hole may extend in a direction that is perpendicular or oblique to an outer circumferential surface and/or an inner circumferential surface of the first fixation element. Preferably, the wall hole is a through hole and communicates with the cylinder hollow space of the first fixation element.

According to an advantageous embodiment, the at least one opening may include a further wall hole, namely a further wall hole formed in the wall of the hollow cylinder. In particular, the further wall hole may extend in a direction that is perpendicular or oblique to an outer circumferential surface and/or an inner circumferential surface of the first fixation element. Providing two wall holes has the advantage that a better control of the first fixation element can be achieved when the flexible strand is passed through the wall holes and the first fixation element is then passed in such a state through the hole of the first bone and the hole of the second bone. Preferably, the further wall hole is a through hole and communicates with the cylinder hollow space of the first fixation element.

Preferably, the wall holes are arranged in a row/on a line in a direction from the first end of the hollow cylinder to the second end of the hollow cylinder. The expression “in a row/on a line” in the aforementioned direction means in particular that the wall holes are arranged one after the other in the aforementioned direction. More preferably, the wall holes may be arranged in a row/on a line parallel to the longitudinal axis. In other words, the wall holes of the first fixation element are more preferably arranged at the same position in a circumferential direction of the hollow cylinder and at different positions in a direction parallel to the longitudinal axis of the hollow cylinder.

Preferably, the wall holes may be arranged symmetrically to a central transverse axis of the hollow cylinder. This configuration has the advantage that the first fixation element may be positioned symmetrically or at least substantially symmetrically to a longitudinal axis of the hole of the second bone, when the second bone is fixed to the first bone by the bone fixation device. However, it is also possible that the wall holes of the first fixation element are arranged asymmetrically to the central transverse axis of the hollow cylinder. The central transverse axis is a transverse axis (axis perpendicular to the inner and/or outer circumferential surface) passing through the centre of the hollow cylinder. The centre of the hollow cylinder is in particular a geometric centre and in particular also a centre of mass of the hollow cylinder.

Preferably, the wall holes are identically formed in terms of shape and/or size. This can simplify the production of the first fixation element. It is understood that the described wall hole(s) is/are opening(s) within the context of the present invention.

It is also possible that more than two wall holes are formed in the wall of the hollow cylinder, the wall holes extending in a direction that is perpendicular or oblique to the outer circumferential surface and/or the inner circumferential surface of the hollow cylinder and being through holes communicating with the cylinder hollow space. Providing more than two wall holes has the advantage that the flexible strand can be passed through the wall holes such that the flexible strand cannot be easily detached from the first fixation element, in particular not by pulling at one of the ends of the flexible strand. In order to achieve this, an end of the flexible strand can be consecutively passed through the wall holes in the order of their arrangement, so that the flexible strand has a wave-like shape when received by the first fixation element. In particular, the more than two wall holes may be arranged in a row/on a line parallel to the longitudinal axis of the hollow cylinder.

The wall hole(s) may preferably have a circular cross-section. It is however also possible that the wall hole(s) have a cross-section of another shape, e.g. an elliptical cross-section.

A minimum distance between neighbouring wall holes preferably lies between 1 mm and 3 mm. The minimum distance is in particular understood as the minimum distance between the contours of the wall holes.

According to an advantageous embodiment, the at least one opening may include a slit in the wall that extends from the first end of the hollow cylinder in the direction from the first end to the second end. It is apparent that the expression “in the direction from the first end to the second end” does not mean that the slit extends up to the second end but rather indicates the direction in which the slit extends. In fact, the slit extends from the first end of the hollow cylinder and to a position within a middle region of the hollow cylinder between the first end and the second end. More preferably, the at least one opening may include a further slit in the wall that extends from the second end of the hollow cylinder in the direction from the second end to the first end. It is apparent that the expression “in the direction from the second end to the first end” does not mean that the slit extends up to the first end but rather indicates the direction in which the slit extends. In fact, the further slit extends from the second end of the hollow cylinder to a position within a middle region of the hollow cylinder between the first end and the second end. If the aforementioned two slits are formed in the wall, it is preferable that they are aligned with each other. This means in particular that the longitudinal axes of the slits are collinear. It is further preferable that the slit(s) extends/extend parallel to the longitudinal axis of the hollow cylinder. Within the context of the present invention, a slit can preferably be understood as an elongated, in particular narrow, through opening (cut).

According to an advantageous embodiment, the at least one opening may include a channel formed through the wall. The channel extends from the first end of the hollow cylinder to the second end of the hollow cylinder. This design has the advantage that the flexible strand can be passed through the channel without being positioned inside the cylinder hollow space of the first fixation element. Thus, if a guiding element is used, over which the first fixation element is guided through the hole of the first bone and the hole of the second bone, passing the first fixation element through the holes can be simplified, as the surgeon does not have to pay attention that the flexible strand does not interfere with the guiding element. Preferably, the channel may extend parallel to the longitudinal axis of the hollow cylinder.

According to an advantageous embodiment, the at least one opening may include a groove formed in an inner circumferential surface of the hollow cylinder in a direction from the first end of the hollow cylinder to the second end of the hollow cylinder. The formation of a groove in the inner circumferential surface has the advantage that the flexible strand can be received in the groove so that the flexible strand may not interfere with a guiding element, in particular guidewire, used for guiding the first fixation element. Preferably, the groove extends parallel to the longitudinal axis of the hollow cylinder.

According to an advantageous configuration, the groove extends from the first end to the second end of the hollow cylinder, i.e. starts at the first end and ends at the second end of the hollow cylinder. According to an alternative advantageous configuration, the groove may communicate with the previously described wall holes, the wall holes being through holes and communicating with the cylinder hollow space. It is understood that in this configuration the groove may not extend from the first end to the second end of the hollow cylinder but may be formed in a middle portion of the hollow cylinder, in particular between the two wall holes.

According to an advantageous embodiment, the at least one opening may include a pocket formed on an inner circumferential surface of the hollow cylinder. The pocket may advantageously communicate with the two above described wall holes formed as through holes in the wall of the hollow cylinder. This design also has the advantage that the flexible strand can be passed through the wall holes and the pocket so that it does not interfere with a guiding element, in particular guidewire, used for guiding the first fixation element. Within the present invention, the pocket may advantageously be understood as a receptacle defining an inner space or cavity.

According to an advantageous embodiment, the at least one opening includes a channel, which extends through the wall of the hollow cylinder, i.e. is formed inside the wall, and starts from the outer circumferential surface of the hollow cylinder and ends at the outer circumferential surface of the hollow cylinder. This design has the advantage that the flexible strand can be passed through the channel so that it does not interfere with a guiding element, in particular guidewire, used for guiding the first fixation element. Preferably, the channel is at least partly, in particular completely, formed as a curved channel.

The longitudinal axis of the hollow cylinder advantageously corresponds to the longitudinal axis of the first fixation element. Accordingly, the central transverse axis of the hollow cylinder advantageously corresponds to the central transverse axis of the first fixation element.

Preferably, an outer edge of a distal end face of the first fixation element is rounded. Due to the rounded outer edge of its distal end face, the first fixation element can be forwarded through the bone hole more easily. Further, the rounded outer edge acts as a non-invasive outer edge. More preferably, the distal end face of the first fixation element is rounded.

Preferably, the first fixation element includes at least one mating element for mating with at least one mating element of a pusher, the pusher being configured for pushing and in particular also for rotating the first fixation element through the hole of the first bone and the hole of the second bone. The mating element of the first fixation element is in particular a proximal mating element, whereas the mating element of the pusher is in particular a distal mating element. For passing the first fixation element through the hole of the first bone and the hole of the second bone, the surgeon places the pusher onto the first fixation element such the at least one mating element of the first fixation element is mated with the at least one mating element of the pusher. Then, according to the needs of the procedure, the surgeon may push and in particular also rotate the pusher so that the first fixation element is passed through and out of the hole of the first bone and the hole of the second bone.

Advantageously, the at least one mating element of the first fixation element has a complementary shape to that of the at least one mating element of the pusher.

The at least one mating element preferably of the first fixation element includes a projection. The projection can in particular be a tongue. Correspondingly, the at least one mating element of the pusher preferably includes a groove that is configured to mate with the projection.

Alternatively or in addition, the at least one mating element of the first fixation element may advantageously include a groove. Correspondingly, the at least one mating element of the pusher may preferably include, alternatively or in addition to a projection, a groove that is configured to mate with the projection of the first fixation element. The projection can in particular be a tongue.

The at least one mating element of the first fixation element is advantageously formed at a proximal end, in particular on a proximal end face, of the hollow cylinder. The at least one mating element of the pusher is advantageously formed at a distal end, in particular on a distal end face, of the pusher.

The at least one mating element of the first fixation element advantageously extends in a direction parallel to the longitudinal axis of the hollow cylinder. The at least one mating element of the pusher advantageously extends in a direction parallel to a longitudinal axis of the pusher. Advantageously, the longitudinal axis of the pusher is parallel to or coincides with the longitudinal axis of the hollow cylinder, when the at least one mating element of the first fixation element is mated with the at least one mating element of the pusher.

Advantageously, the flexible strand is passed through the at least one opening of the first fixation element. In particular, the flexible strand may be already passed through the at least one opening of the first fixation element. In other words, the bone fixation device may preferably be prefabricated such that the first fixation element is preloaded on the flexible strand or, alternatively formulated, such that the flexible strand is received by the first fixation device, in particular by the at least one opening. In particular, the flexible strand may advantageously be fixed to the first fixation element.

According to an alternative advantageous configuration, the flexible strand may be provided separately from the first fixation element in the bone fixation device (not already passed through the at least one opening). In this case, the surgeon passes the flexible strand though the at least one opening of the first fixation element for bone fixation before the first fixation element is passed through the hole of the first bone and the hole of the second bone.

When the flexible strand is in a passed-through state, either already passed through the at least one opening of the first fixation element or after the surgeon has passed it through the at least one opening of the first fixation element, part of the flexible strand is received in the at least one opening.

Preferably, the bone fixation device further includes a second fixation element configured for engaging the first bone and for securing the flexible strand. The expression that the second fixation element is configured for securing the flexible strand means in particular that the shape of the second fixation element is such and/or that the second fixation element has a securing means such that the flexible strand is securable to the second fixation element. Preferably, securing of the flexible strand to the second fixation element occurs by tying the flexible strand to the second fixation element. For example, the second fixation element may have the shape of a butterfly such that the flexible strand can be tied to the second fixation element. Securing, in particular tying, the flexible strand to the second fixation element preferably includes the option of securing a first strand region (in particular, a first strand end region) and a second strand region (in particular, a first strand end region) of the flexible strand directly to the second fixation element, the option of securing the first strand region directly to the second strand region of the flexible strand in such a way that the second fixation element engages the first bone for fixation of the second bone to the first bone or a combination of these two options.

The second fixation element may preferably be formed as a plate-shaped element. This has the advantage of a simple design. The second fixation element can, however, be formed in any shape that can serve the purpose of engaging the first bone and securing the flexible strand for fixing the first bone and the second bone to each other.

The term “plate-shaped” particularly describes in the framework of the present invention a shape with one dimension that is much smaller than the other dimension(s) that is/are used to geometrically describe the shape. This is in particular/preferably the case if a ratio of the much smaller dimension to the other dimension(s) lies between 1 to 15 and 1 to 10. A plate-shaped element advantageously has two main surfaces, which are preferably parallel to each other. If a plate-shaped element has a circular shape, the thickness is much smaller than its diameter.

The second fixation element preferably includes at least one hole for receiving the flexible strand. In particular, the flexible strand may be passed through the at least one hole and secured, in particular tied, to the second fixation element. Within the context of the present invention, the expression that the at least one hole of the second fixation element is configured for receiving the flexible strand means in particular that the at least one hole is configured such that the flexible strand can pass through the at least one hole.

More preferably, the second fixation element includes two holes for receiving the flexible strand. It is also possible that the second fixation element is provided with more than two holes for receiving the flexible strand.

The at least one hole, in particular the two holes, of the second fixation element is/are advantageously through holes. In order to fix the first bone and the second bone to each other, the flexible strand may be passed through the two holes and secured, in particular tied, to the second fixation element, more specifically to a region of the second fixation element between the two holes. To this end, the aforementioned first strand region and second strand region can be tied to each other and/or to the second fixation element, more specifically to a region of the second fixation element between the two holes.

The at least one hole of the second fixation element advantageously extends in a direction of the thickness of the second fixation element. A hole extending in a direction of the thickness of the second fixation element includes a hole that extends in a direction that is perpendicular or oblique to a main surface of the second fixation element or in other words in a direction that is parallel or oblique to a normal vector of a main surface of the first fixation element.

Within the context of the present invention, a fixation element that is formed as a plate-shaped element and includes at least one hole as mentioned above can also be characterized as a button or surgical button. Within the context of the present invention, the second fixation element being formed as a button can also be described as an anchoring button, as it is used for anchoring the first bone when the first fixation element has been passed through the hole of the first bone and the hole of the second bone and engages the second bone.

The second fixation element preferably has a circular cross-section with a diameter that is larger than or equal to 10 mm and/or smaller than or equal to 15 mm, in particular a diameter that lies between 10 mm and 15 mm. However, it is also possible that the second fixation element has a cross-section of a different shape. For example, the cross-section of the second fixation element can be elliptical or that of a butterfly.

Preferably, the flexible strand forms a closed loop, when the first bone and the second bone are fixed to each other.

Advantageously, the second fixation element may be preloaded on the flexible strand. In particular, the flexible stand may be prefabricated such that it is already passed through two holes of the second fixation element.

Preferably, the second fixation element may be loaded, more preferably preloaded, on the flexible strand, in particular the second fixation element may be passed through two holes of the second fixation element, such that pulling the flexible strand causes the second fixation element to move in a direction opposite to the pulling direction (i.e. the second fixation to move towards the first bone), in particular such that pulling the flexible strand in the pulling direction causes the second fixation element to engage the first bone. This means that in this configuration the second fixation element is preferably movably loaded, more preferably movably preloaded, on the flexible strand. It is also possible that the flexible strand is configured such that it shortens when pulled, thereby causing the second fixation element to move in a direction opposite to the pulling direction (i.e. be moved towards the first bone), in particular such that pulling the flexible strand in the pulling direction causes the second fixation element to engage the first bone.

It is understood that the first fixation element and the flexible strand as well the second fixation element, if provided, are each made from a biocompatible material.

The first fixation element may preferably be made of a metal material or polymer material. The second fixation element may preferably be made of a metal material or polymer material.

According to an advantageous embodiment, the bone fixation device may preferably be configured for bone fixation in a lesser trochanteric fragment, in which a lesser trochanteric fragment is detached from a femur bone. Here, the aforementioned first bone is the femur bone and the aforementioned second bone is the lesser trochanteric fragment. The hole of the first bone, namely the femur bone, can be characterized as a femur bone hole and the hole of the second bone, namely the lesser trochanteric fragment, as a lesser trochanteric fragment hole. Thus, here, the first fixation element is configured/adapted for passing through a femur bone hole and a lesser trochanteric fragment hole and for engaging the lesser trochanteric fragment. The lesser trochanter fracture can be an isolated fracture or a secondary fracture. In the latter case, the main feature may be an intertrochanteric fracture. With the bone fixation device of the present invention, healing of the lesser trochanter fracture and a satisfactory restoration of the function of the hip of a patient with reduced pain may be achieved. Another advantage is that, in the case of the occurrence of the lesser trochanter fracture as a component of another type of hip fracture, i.e. as a secondary fracture, the stabilization of the lesser trochanteric fragment achieved by means of the bone fixation device of the present invention positively affects the healing of the other type of hip fracture, i.e. the main fracture, as well. Particularly in the case where the lesser trochanter fracture is a component of an intertrochanteric fracture, the bone fixation device on one hand achieves the treatment of the lesser trochanter fracture and on the other hand increases the success of the treatment of the intertrochanteric fracture.

According to an advantageous embodiment, the bone fixation device may preferably be configured for bone fixation in an acromioclavicular joint dislocation. The acromioclavicular joint, or ACJ joint, is a joint at the top of the shoulder. It is the junction between the acromion and the clavicle. With direct force, normally a fall onto the tip of the shoulder, the joint can be dislocated and become painful and unstable. Here, the first bone and the second bone that need to be fixed to each other in order for the acromioclavicular joint dislocation to be repaired are the clavicle and the coracoid process, respectively. The hole of the first bone, namely the clavicle, can be characterized as a clavicular hole and the hole of the second bone, namely the coracoid process, as a coracoid process hole. Thus, here, the first fixation element is configured/adapted for passing through a clavicle hole and a coracoid process hole and for engaging the coracoid process. By using the bone fixation device according to the present invention, the surgical technique can be easy with minimal surgical approach, shorter operative time and protection of neurovascular bundle at the same time. The clavicle is reduced, the ligaments of the joint can be heeled and, in addition, hardware removal is not necessary.

The first fixation element and/or the second fixation element, and in particular also the flexible strand can me made of a biodegradable material. Thus, the bone fixation device can provide a temporary support until the first bone and the second bone in the case of a bone fracture are attached to each other and the joint function in the case of a joint dislocation is restored.

The present invention further refers to an arrangement including a previously described bone fixation device. The advantages presented above in view of the previously described bone fixation device apply also here. The arrangement can particularly be understood as a kit. The arrangement advantageously includes one or more instruments (tools) to be used during a bone fixation method for fixation of a first bone and a second bone to each other in a bone fracture and/or a joint dislocation. In the following, such instruments will be presented.

The arrangement preferably includes a pusher for pushing, and in particular also for rotating, the first fixation element of the bone fixation device through the hole of the first bone and the hole of the second bone.

Preferably, the pusher includes at least one mating element that is configured to mate with at least one mating element of the first fixation element. The pusher advantageously corresponds to the pusher previously described. This means that the possible advantageous configurations of the at least one mating element of the pusher apply here too. Similarly, the possible advantageous configurations of the at least one mating element of the first fixation element apply here too.

Preferably, the pusher has a pusher hollow space, in which the flexible strand is preloaded and kept in tension. This means that the pusher may preferably be configured such that the flexible strand is preloaded and kept in tension inside the pusher hollow space. The pusher can also be described as cannulated due to its pusher hollow space.

The pusher may preferably be formed as a cylinder. In particular, the pusher may be formed as a hollow cylinder.

The pusher may preferably be provided with pusher distance/depth marks. Based on the pusher distance/depth marks, the surgeon can check how deep the first fixation element has been pushed inside the hole of the first bone and/or the hole of the second bone.

The arrangement may preferably include a protective sleeve. The protective sleeve is advantageously hollow. In particular, the protective sleeve may be formed as a hollow cylinder. The protective sleeve is used for passing the first fixation element therethrough and advancing it up to the hole of the first bone. To this end, the protective sleeve, in particular its distal end is advantageously placed against the first bone. For example, the protective sleeve can be placed against the femur bone, in particular its lateral cortex, during bone fixation in a lesser trochanter fracture or against the clavicle, in particular its cranial cortex, during bone fixation in an acromioclavicular joint dislocation. As the first fixation element is passed through the hollow protective sleeve, it is not exposed to the surrounding, in particular muscle, tissues, of the first bone, through which the bone fixation element has to go for accessing the hole of the first bone. Thus, the use of the protective sleeve has the advantage that it defines an area unobstructed from the surrounding, in particular muscle, tissues, for the first fixation element. At the same time, the protective sleeve provides enhanced protection for the surrounding tissues. Thus, the surgeon can more easily perform bone fixation of the first bone and the second bone to each other.

Advantageously, a handle is attached to the protective sleeve. Thereby, the surgeon can have a better grip of and control over the protective sleeve while it is placed against the first bone as described above.

For achieving a more stable placement of the protective sleeve against the first bone, the distal end of the protective sleeve may be provided with gripping means for gripping onto the first bone, in particular the lateral cortex of the femur bone during bone fixation in a lesser trochanter fracture or the cranial cortex of the clavicle during bone fixation in an acromioclavicular joint dislocation. The gripping means may preferably include one or more teeth.

Advantageously, any instrument to be used for the bone fixation may be configured to be passed through the protective sleeve. Accordingly, the protective sleeve can advantageously be configured such any instrument used for the bone fixation can pass through it.

The arrangement preferably includes a Kirschner wire (K-wire), more preferably a threaded Kirschner wire.

The Kirschner wire is advantageously configured to be passed through the first bone and the second bone. The Kirschner wire is advantageously used for securing the second bone. By the advancement of the Kirschner wire through the first bone and the second bone, a preliminary hole in the first bone and a preliminary hole in the second bone are formed. Within the context of the present invention, the preliminary hole of the first bone and the preliminary hole of the second bone advantageously define a preliminary bone hole. For bone fixation in a lesser trochanter fracture, the preliminary hole of the first bone can be characterized as a preliminary lesser trochanteric fragment hole and the preliminary hole of the second bone as a preliminary femur bone hole. For bone fixation in an acromioclavicular joint dislocation, the preliminary hole of the first bone can be characterized as a preliminary clavicular hole and the preliminary hole of the second bone as a preliminary coracoid process hole. Further, the Kirschner wire can advantageously be used for passing a cannulated drill over the Kirschner wire for drilling the bone hole. The Kirschner wire preferably has a diameter between 1.7 mm and 2.3 mm, more preferably between 1.9 mm and 2.1 mm, in particular 2 mm.

The arrangement preferably includes a drill for drilling the hole of the first bone and/or the hole of the second bone.

The drill is preferably provided with drill distance/depth marks. The drill distance/depth marks help the surgeon know how much the drill has been advanced inside the first bone and the second bone and consequently the depth of the corresponding drilled holes at any time during the procedure. Thus, the surgeon can accurately drill the needed holes through the first bone and the second bone.

Further, the drill is advantageously configured such that it can be inserted in the protective sleeve, if such a protective sleeve is used.

More preferably, the drill is a cannulated drill. The cannulated drill has the advantage that it can be passed over the Kirschner wire, the latter acting as a guiding element for the cannulated drill.

The arrangement preferably includes a guide sleeve for guiding the Kirschner wire, while the Kirschner wire is being passed through the first bone and the second bone. To this end, the guide sleeve preferably has a guide sleeve hollow space for guiding the Kirschner wire therethrough. The Kirschner wire can in particular be guided through the guide sleeve hollow space in a sliding manner. Preferably, a shape and a size of the cross-section of the guide sleeve hollow space is substantially the same with a shape and a size of the cross-section of the Kirschner wire, respectively. The guide sleeve hollow space is preferably a longitudinal channel. The guide sleeve hollow space may preferably have a diameter between 1.7 mm and 2.3 mm, more preferably between 1.9 mm and 2.1 mm, in particular 2 mm.

The guide sleeve is preferably configured such that it is insertable in the protective sleeve. In particular, the guide sleeve may preferably include a main sleeve body that is insertable in the protective sleeve and a stop means for securing the position of the main sleeve body relative to the protective sleeve in an axial direction, and preferably also in a circumferential direction. The stop means may advantageously be configured such that the guide sleeve, in particular the stop means, abuts against a proximal end face of the protective sleeve, when the guide sleeve, in particular the main sleeve body, is inserted in the protective sleeve. Preferably, the stop means may be formed as or include a flange.

The arrangement may preferably include a ruler.

The ruler is advantageously configured/used for deriving a distance between the proximal cortex of the first bone, or more particularly a proximal end of the hole of the first bone, and a distal end of the Kirschner wire. If the Kirschner wire is advanced such that its distal end coincides with a distal cortex of the second bone, or more particularly with a distal end of the hole of the second bone, the distance between the proximal cortex of the first bone and the distal end of the Kirschner wire corresponds to a distance between the proximal cortex of the first bone and the distal end of the second bone. In case of bone fixation in a lesser trochanter fracture, the proximal cortex of the first bone in particular corresponds to a lateral cortex of the femur bone, the proximal end of the hole of the first bone to a lateral end of the femur bone hole and the distal end of the hole of the second bone to a medial end of the lesser trochanteric fragment bone hole. In case of bone fixation in an acromioclavicular joint dislocation, the proximal cortex of the first bone in particular corresponds to a cranial cortex of the clavicle, the proximal end of the hole of the first bone to a cranial end of the clavicular hole and the distal end of the hole of the second bone to a caudal end of the clavicular hole.

To this end, the ruler advantageously has a longitudinal ruler channel to receive the Kirschner wire. The ruler is provided with ruler distance marks.

According to a first advantageous configuration, the ruler is configured/used for measuring a distance between the proximal cortex of the first bone and a proximal end of the Kirschner wire. In other words, the ruler is advantageously configured/used for measuring a distance of the Kirschner wire being outside the first bone and extending outwards from the proximal cortex of the first bone. Thus, as the overall length of the Kirschner wire is known and the distance between the proximal cortex of the first bone and the proximal end of the Kirschner wire is measured by the ruler, the distance between the proximal cortex of the first bone and the distal end of the Kirschner wire and in particular a distance between the proximal cortex of the first bone and a distal end of the second bone, when the distal end of the Kirschner wire coincides with the distal end of the second bone, can be derived. With this configuration/use of the ruler, the previously described guide sleeve is either not used or a guide sleeve is used that is configured such that it can guide the Kirschner wire and also receive the ruler in its ruler hollow space. In the latter case, the ruler is configured such that it can be inserted in the guide sleeve.

According to a second advantageous configuration, the ruler is configured/used for measuring a distance between a proximal end of the previously described guide sleeve and a proximal end of the Kirschner wire. In other words, the ruler is advantageously configured for measuring a distance of the Kirschner wire that is outside the first bone and the guide sleeve, and extends outwards from the guide sleeve. Thus, as the overall length of the Kirschner wire and the length of the guide sleeve are known, and the distance between the proximal end of the guide sleeve and the proximal end of the Kirschner wire is measured by the ruler, the distance between the proximal cortex of the first bone and the distal end of the Kirschner wire and in particular a distance between the proximal cortex of the first bone and a distal end of the second bone, when the distal end of the Kirschner wire coincides with the distal end of the second bone, can be derived. The ruler according to this configuration can advantageously be used with the previously described guide sleeve.

During bone fixation, the surgeon places, in particular slides, the ruler over the Kirschner wire so that the Kirschner wire is received in the longitudinal ruler channel, and advances the ruler until the ruler has reached the proximal cortex of the first bone, if the first advantageous configuration of the ruler is used, or until the ruler has reached the proximal end of the guide sleeve, if the second advantageous configuration of the ruler is used.

The ruler is advantageously configured such that it can be inserted in the protective sleeve, if such a protective sleeve is used.

The arrangement may preferably include a guidewire, in particular an atraumatic wire. An atraumatic wire is in particular a guidewire with an atraumatic tip, in particular a rounded tip. The guidewire is advantageously configured for guiding the first fixation element. To this end, the first fixation element is passed over the guidewire and advanced through the hole of the first bone and the hole of the second bone, preferably by being pushed by the pusher described above.

It is noted that the terms “distal” and “proximal” with respect to a feature of any component of the bone fixation device or the arrangement mean that a distal feature is closer to the patient than the proximal feature is to the patient, when/while the component of the bone fixation device or the arrangement is being inserted in the patient's body or used during the bone fixation. Within the context of the present invention, in particular, a proximal feature of a component of the bone fixation device can also be characterized as a first feature and the corresponding distal feature of the component of the bone fixation device as a second feature.

On the other hand, a proximal region and a distal region of a bone mean within the context of the present invention that the proximal region is closer to the surgeon than the distal region is to the surgeon. For example, a proximal cortex of the first bone is the cortex of the first bone that is closer to the surgeon than the distal cortex.

The pusher, the protective sleeve, the Kirschner wire, the drill, the guide sleeve, the ruler and the guidewire described above correspond to instruments to be used during bone fixation. Thus, the arrangement may preferably include the previously described bone fixation device and at least one instrument of the following instruments: the pusher, the protective sleeve, the Kirschner wire, the drill, the guide sleeve, the ruler and the guidewire as described above.

It is noted that the bone fixation device can be sold without the flexible strand.

Further disclosed is a bone fixation method for fixation of a first bone and a second bone to each other in a bone fracture, in particular a lesser trochanter fracture, by means of a bone fixation device. The bone fixation device includes a first fixation element configured for passing through a hole of the first bone and a hole of the second bone and for engaging the second bone and a flexible strand. The first fixation element is further configured for receiving the flexible strand. The bone fixation method includes the steps of:
forming a hole in the first bone and a hole in the second bone,
passing, in particular pushing, the first fixation element, with the flexible strand received by the first fixation element, through the hole of the first bone and through the hole of the second bone so that the first fixation element exits the hole of the second bone,
engaging the second bone by means of the first fixation element, and
fixing the first bone and the second bone to each other by means of the flexible strand.

In the bone fixation method for fixation of a first bone and a second bone to each other in a bone fracture, in particular a lesser trochanter fracture, the first fixation element may according to an advantageous embodiment be formed as a plate-shaped element with at least one through hole, in particular two through holes, for receiving the flexible strand, in particular through which the flexible strand is passed. Here, the flexible strand is passed through the at least one through hole, in particular the two through holes, such that the flexible strand is received by the first fixation element. In particular, the first fixation element may be elongated. In other words, the first fixation element may be a button, in particular an elongated button. “Elongated” means in particular that the first fixation element extends in a direction more than the other two directions. The bone fixation method may advantageously include the step of loading the first fixation element on the flexible strand, in particular by passing the flexible strand through the at least one through hole of the first fixation element. It is understood that this step is advantageously executed before passing the first fixation element through the hole of the first bone and the hole of the second bone. Alternatively, the first fixation element may be preloaded on the flexible strand, in particular the flexible strand can advantageously already be passed through the at least one through hole of the first fixation element. The flexible strand may particularly be fixed, in particularly prefixed, to the first fixation element.

In the bone fixation method for fixation of a first bone and a second bone to each other in a bone fracture, in particular a lesser trochanter fracture, the first fixation element may according to an alternative advantageous embodiment include a hollow cylinder, wherein the hollow cylinder has a wall defining a cylinder hollow space and extends in a direction of a longitudinal axis from a first end to a second end.

Further disclosed is a bone fixation method for fixation of a first bone and a second bone to each other in a joint dislocation, in particular in an acromioclavicular joint dislocation, by means of a bone fixation device. The bone fixation includes a first fixation element and a flexible strand. The first fixation element is configured for passing through a hole of the first bone and a hole of the second bone and for engaging the second bone. The first fixation element includes a hollow cylinder, in particular is formed as a hollow cylinder, wherein the hollow cylinder extends in a direction of a longitudinal axis from a first end to a second end and has a wall defining a cylinder hollow space. The bone fixation method including the steps of:
forming a hole in the first bone and a hole in the second bone,
passing, in particular pushing, the first fixation element, with the flexible strand received by the first fixation element, through the hole of the first bone and through the hole of the second bone so that the first fixation element exits the hole of the second bone,
engaging the second bone by means of the first fixation element, and
fixing the first bone and the second bone to each other by means of the flexible strand.

Within the framework of the present invention, the expression “with the flexible strand received by the first fixation element” can in particular be replaced by the expression “with the first fixation element loaded on the flexible strand”.

In the following description, features, explanations etc. that relate to the first fixation element including the hollow cylinder apply to both the method for fixation of a first bone and a second bone to each other in a bone fracture and the method for fixation of a first bone and a second bone to each other in a joint dislocation, when a bone fixation device with such a first fixation element is used.

In particular, the first fixation element is passed first through the hole of the first bone and then through the hole of the second bone. In other words, the first bone is in particular the first to be accessed by the surgeon. Exiting of the first fixation element means in particular that the first fixation element has completely exited/come out the hole of the second bone. In other words, this means that a proximal end face of the first fixation element has exited/come out of the hole of the second bone.

According to an embodiment, fixing of the first bone and the second bone to each other can be achieved by forming a bone bridge in the first bone and attaching, in particular tying, the flexible strand to the bone bridge, as previously described. According to an alternative advantageous embodiment, the bone fixation device may preferably further include a second fixation element configured for securing a flexible strand. The method preferably further includes the step of engaging the first bone by means of the second fixation element, and the step of fixing the first bone and the second bone to each other by securing the flexible strand to the second fixation element.

In the configuration, where the first fixation element includes a hollow cylinder, in particular is formed as a hollow cylinder, passing the first fixation element through the hole of the first bone and then through the hole of the second bone so that the first fixation element exits the hole of the second bone in particular means passing the hollow cylinder through the hole of the first bone and through the hole of the second bone so that the hollow cylinder exits the hole of the second bone. The first fixation element is advantageously passed through the hole of the first bone and the hole of the second bone by passing the first fixation element over a guidewire for guiding the first fixation element. The guidewire is inserted in the hole of the first bone and the hole of the second bone. Preferably, the guidewire is an atraumatic wire. Advantageously, the guidewire is removed after the first fixation element has exited the hole of the second bone.

The aforementioned configurations of the bone fixation device with the first fixation element including a hollow cylinder, in particular the configurations of this first fixation element, as well as considerations and advantages with respect to the bone fixation device, in particular the first fixation element, and the above described arrangement can be combined with and/or apply to the disclosed bone fixation methods for bone fixation of a first bone and a second bone to each other in a bone fracture and in a joint dislocation.

Thus, the first fixation element may preferably include at least one opening that is configured for receiving the flexible strand, wherein the flexible strand is passed through the at least one opening. The bone fixation method may advantageously include the step of passing the flexible strand through the at least one opening of the first fixation element. It is understood that this step is advantageously executed before passing the first fixation element through the hole of the first bone and the hole of the second bone. Alternatively, the flexible strand can advantageously already be passed through the at least one opening of the first fixation element, and may particularly be already fixed to the first fixation element.

For example, the at least one opening can include the above-described elements with regard to the description of the bone fixation device of the present invention.

As described above, the at least one opening may advantageously include two wall holes formed in the wall of the hollow cylinder. The wall holes may extend in a direction that is perpendicular or oblique to an outer circumferential surface and/or an inner circumferential surface of the hollow cylinder. The wall holes may be through holes and communicate with the cylinder hollow space. The wall holes may be arranged in a row in a direction from the first end to the second end, in particular in a direction parallel to the longitudinal axis. The wall holes may be arranged symmetrically to a central transverse axis of the hollow cylinder.

In this configuration, the flexible strand is received by the first fixation element in that the flexible strand is passed through the two wall holes. In the received state of the flexible strand, part of the flexible strand is arranged inside the cylinder hollow space of the first fixation element. To this end, a strand end of the flexible strand may be passed through the first one of the wall holes, then through the cylinder hollow space of the first fixation element and finally through the second one of the wall holes.

Further as described above, the at least one opening may advantageously include a slit in the wall that extends from the first end of the hollow cylinder in the direction from the first end to the second end, and a further slit in the wall that extends from the second end of the hollow cylinder in the direction from the second end to the first end.

In this configuration, the flexible strand may be received by the first fixation element by passing the flexible strand through the slits. In the received state of the flexible strand, part of the flexible strand is arranged inside the cylinder hollow space of the first fixation element. To this end, a strand end of the flexible strand may be passed through the first one of slits, the cylinder hollow space of the first fixation element and the second one of the slits.

If the at least one opening includes a channel formed inside the wall and extending from the first end to the second end of the cylinder as described above, the flexible strand may be received by the first fixation element by passing the flexible strand through the channel.

In this configuration, part of the flexible strand is arranged inside the channel, while the rest of the flexible strand is outside the channel. To this end, a strand end of the flexible strand may be passed through the channel until it exits the channel. Here, the flexible strand is not arranged inside the cylinder hollow space of the first fixation element.

If the at least one opening includes a groove formed in an inner circumferential surface of the hollow cylinder and extending in a direction from the first end to the second end, in particular from the first end to the second end, as described above, the flexible strand may be received by the first fixation element by passing the flexible strand through the groove. In this configuration, part of the flexible strand can be arranged inside the groove.

If the at least one opening includes a pocket formed on an inner circumferential surface of the hollow cylinder, the flexible strand may be received by the first fixation element by passing the flexible strand through the pocket. In this configuration, part of the flexible strand can be arranged inside the pocket.

If the at last one opening includes a channel that is formed inside the wall of the hollow cylinder and starts from and ends at the outer circumferential surface of the hollow cylinder, the flexible strand may be received by the first fixation element by passing the flexible strand through the channel.

If the second fixation element includes at least one through hole for receiving the flexible strand as described above, the method preferably includes the step of passing the flexible strand through the at least one through hole of the second fixation element.

The step of passing the flexible strand through the at least one through hole of the second fixation element preferably includes passing a first strand end of the flexible strand and a second strand end of the flexible strand through the at least one through hole. If the second fixation element includes two through holes, the step of passing the flexible strand though the through holes of the second fixation element preferably includes passing the first strand end of the flexible strand through the first through hole and the second strand end of the flexible strand through the second through hole.

The step of securing the flexible strand to the second fixation element may preferably include securing the first strand region and the second strand region of the flexible strand to the second fixation element, in particular by fixing any of the first strand region and second strand region directly to the second fixation element and/or by connecting, in particular tying, the first strand region and the second strand region to each other. Most preferably, the first strand region and the second strand region of the flexible strand are connected to each other so that the flexible strand forms a loop around the region between the through holes.

Advantageously, the step of passing, in particular pushing, the first fixation element through the hole of the first bone and the hole of the second bone includes pushing the first fixation element through the hole of the first bone and the hole of the second bone by means of a pusher. The pusher preferably includes at least one mating element and the first fixation element includes at least one mating element being mated with the at least one mating element of the pusher for pushing the first fixation element. In other words, the at least one mating element of the first fixation element and the least one mating element of the pusher are mated with each other and the first fixation element is pushed through the first-bone hole and the second-bone hole.

Preferably, the first fixation element is pushed through the hole of the first bone and the hole of the second bone while keeping the flexible strand in tension. Thus, a better control of the first fixation element can be achieved. In particular, the flexible strand may be kept in tension by being loaded, in particular preloaded, in a pusher hollow space.

The tension is advantageously released when the first fixation element has exited the hole of the second bone. This can preferably be done by releasing the flexible strand from the pusher.

The step of forming the hole of the first bone and the hole of the second bone preferably includes the step of advancing a Kirschner wire, preferably a threaded Kirschner wire, through the first bone and the second bone.

The step of forming the hole of the first bone and the hole of the second bone further includes the step of passing a cannulated drill over the Kirschner wire for drilling the hole of the first bone and the hole of the second bone.

Advantageously, while maintaining the cannulated drill in place, the Kirschner wire is removed after the hole of the first bone and the hole of the second bone have been formed. Advantageously, a guidewire, in particular an atraumatic wire, for guiding the first fixation element is then inserted in the cannulated drill. Then, the cannulated drill is removed from the hole of the first bone and the hole of the second bone.

Preferably, the Kirschner wire is guided through a guide sleeve, which is directly placed against the proximal cortex of the first bone or inserted in a protective sleeve that is directly placed against the proximal cortex of the first bone. In particular, the guide sleeve is removed before passing the cannulated drill over the Kirschner wire.

Preferably, for engaging the second bone by means of the first fixation element, the flexible strand is pulled after the first fixation element has exited the hole of the second bone. In particular, the flexible strand is pulled in a pulling direction, wherein the pulling direction is opposite to the direction of passing, in particular pushing, to the first fixation element through the hole of the first bone and the hole of the second hole.

Preferably, for engaging the first bone by means of the second fixation element, the second fixation element is placed on the first bone. It is understood that the second fixation element is placed on the first bone with the flexible strand being received by the second fixation element. This can be done manually by directly placing the second fixation element on the first bone. Alternatively, if the second fixation element is loaded on the flexible strand such that pulling the flexible strand causes the second fixation element to move in a direction opposite to the pulling direction for engaging the first bone and/or if the flexible strand is configured such that pulling the flexible strand when received by the first fixation element and the second fixation element causes the second fixation element to move in a direction opposite to the pulling direction for engaging the first bone, the flexible strand is preferably pulled for engaging the first bone by means of the second fixation element.

Preferably, the bone fixation method can be applied to a patient, in particular a human.

The bone fixation method can, however, also be applied to a cadaver, in particular a human cadaver, or a surgical phantom for training purposes of the surgeons. The human cadaver may include a human cadaveric femur bone and a human cadaveric lesser trochanteric fragment, corresponding to the first bone and second bone, respectively. The surgical phantom may include/represent a human femur bone and a human lesser trochanteric fragment, corresponding to the first bone and second bone, respectively. The phantom may include/represent a human clavicle and coracoid process, corresponding to the first bone and second bone, respectively.

The bone fixation method may preferably include performing radiographic imaging for observing the surgical site, in particular for observing/defining a position of the components of the bone fixation device, in particular a position of the first fixation element of the bone fixation device.

Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The accompanying drawings, which are incorporated in and constitute part of this specification, illustrate embodiments of the invention and together with the description, serve to explain the principles of the invention. The embodiments illustrated herein are presently preferred, it being understood, however, that the invention is not limited to the precise arrangements and instrumentalities shown, wherein:

FIG. 1 is a schematic perspective view of a bone fixation device according to a first embodiment.

FIG. 2 is a schematic perspective view of a pusher to be used in a bone fixation method using the bone fixation device according to the first embodiment.

FIG. 3 is a schematic perspective view of a protective sleeve to be used in a bone fixation method using the bone fixation device according to the first embodiment.

FIG. 4 is a schematic perspective view of a Kirschner wire to be used in a bone fixation method using the bone fixation device according to the first embodiment.

FIG. 5 is a schematic perspective view of a guide sleeve to be used in a bone fixation method using the bone fixation device according to the first embodiment.

FIG. 6 is a schematic perspective view of a ruler to be used in a bone fixation method using the bone fixation device according to the first embodiment.

FIG. 7 is a schematic perspective view of a drill to be used in a bone fixation method using the bone fixation device according to the first embodiment.

FIG. 8 is a schematic perspective view of a guidewire to be used in a bone fixation method using the bone fixation device according to the first embodiment.

FIG. 9 is a schematic perspective view of a first step of a bone fixation method according to a first embodiment for fixing a first bone and a second bone to each other in the case of a lesser trochanter fracture using the bone fixation device of FIG. 1.

FIG. 10 is a schematic perspective view of a second step of the bone fixation method according to the first embodiment using the bone fixation device of FIG. 1.

FIG. 11 is a schematic perspective view of a third step of the bone fixation method according to the first embodiment using the bone fixation device of FIG. 1.

FIG. 12 is a schematic perspective view of a fourth step of the bone fixation method according to the first embodiment using the bone fixation device of FIG. 1.

FIG. 13 is a schematic perspective view of a sixth step of the bone fixation method according to the first embodiment using the bone fixation device of FIG. 1.

FIG. 14 is a schematic perspective view of a seventh step of the bone fixation method according to the first embodiment using the bone fixation device of FIG. 1.

FIG. 15 is a schematic perspective view of an eighth step of the bone fixation method according to the first embodiment using the bone fixation device of FIG. 1.

FIG. 16 is a schematic perspective view of a ninth step of the bone fixation method according to the first embodiment using the bone fixation device of FIG. 1.

FIG. 17 is a schematic perspective view of a state in which the first bone and the second bone are fixed to each other by performing the bone fixation method according to the first embodiment using the bone fixation device of FIG. 1.

FIG. 18 is a schematic perspective view of a first phase of a bone fixation method according to a second embodiment for fixing a first bone and a second bone to each other in the case of an acromioclavicular joint dislocation using the bone fixation device of FIG. 1.

FIG. 19 is a schematic perspective view of a second phase of the bone fixation method according to the second embodiment using the bone fixation device of FIG. 1.

FIG. 20 is a schematic perspective view of a third phase of the bone fixation method according to the second embodiment using the bone fixation device of FIG. 1.

FIG. 21 is a schematic perspective view of a fourth phase of the bone fixation method according to the second embodiment using the bone fixation device of FIG. 1.

FIG. 22 is a schematic perspective view of a sixth phase of the bone fixation method according to the second embodiment using the bone fixation device of FIG. 1.

FIG. 23 is a schematic perspective view of a seventh phase of the bone fixation method according to the second embodiment using the bone fixation device of FIG. 1.

FIG. 24 shows a schematic perspective view of a state in which the first bone and the second bone are fixed to each other by performing the bone fixation method according to the second embodiment using the bone fixation device of FIG. 1.

FIG. 25 is a schematic cross-sectional view of a portion of a bone fixation device according to a second embodiment of the present invention as seen from the front.

FIG. 26 shows a perspective view of a portion of a bone fixation device according to a third embodiment.

FIG. 27 shows a cross-sectional view of a portion of a bone fixation device according to a fourth embodiment as seen from the front.

FIG. 28 shows a cross-sectional view of a portion of a bone fixation device according to a fifth embodiment as seen from the front.

FIG. 29 shows a cross-sectional view of a portion of a bone fixation device according to a sixth embodiment as seen from the back.

FIG. 30 shows a perspective view of a portion of a bone fixation device according to a seventh embodiment.

FIG. 31 shows a portion of a bone fixation device for bone fixation in a lesser trochanter fracture that can be used in a bone fixation method according to a third embodiment.

### DETAILED DESCRIPTION OF THE INVENTION

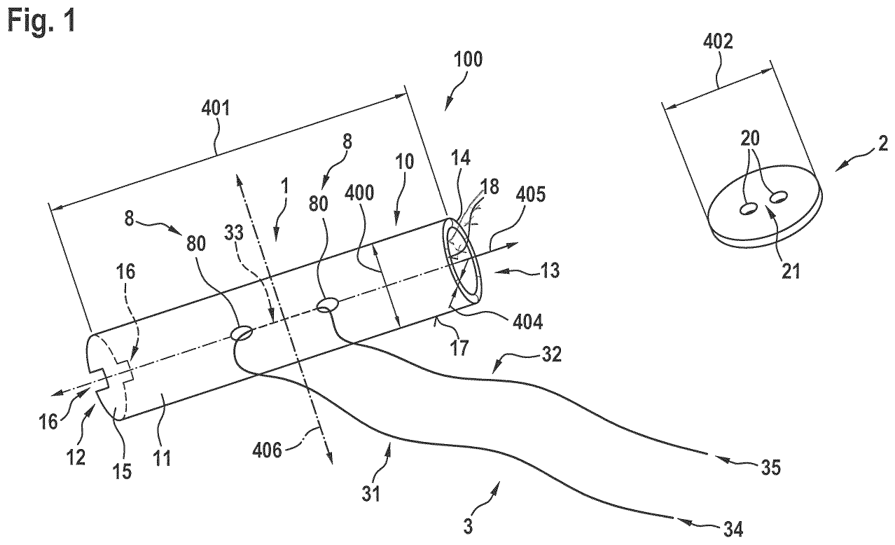

In the following, a bone fixation device 100 according to a first embodiment of the present invention is described in detail by taking reference to FIG. 1.

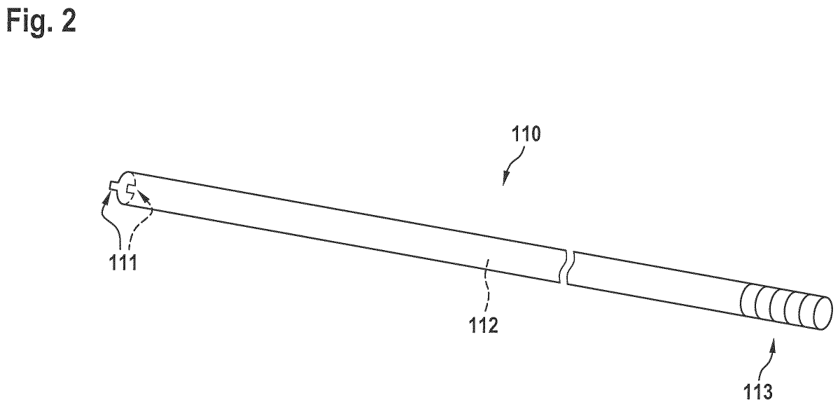

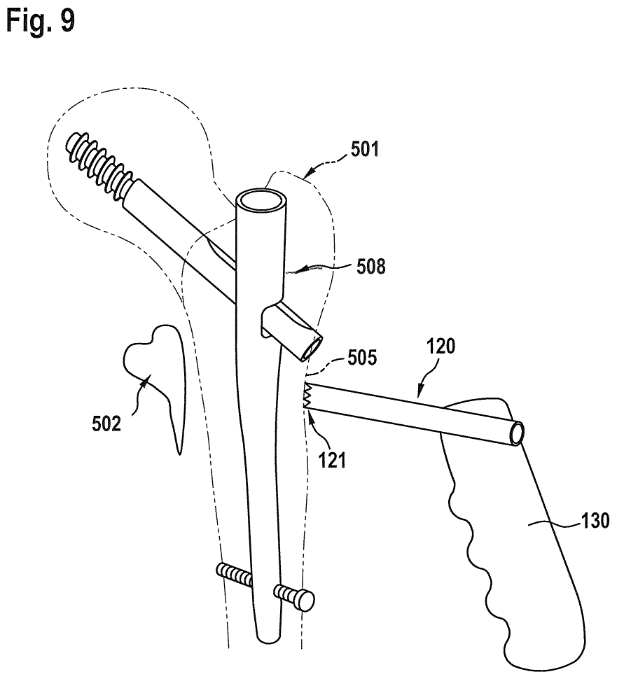

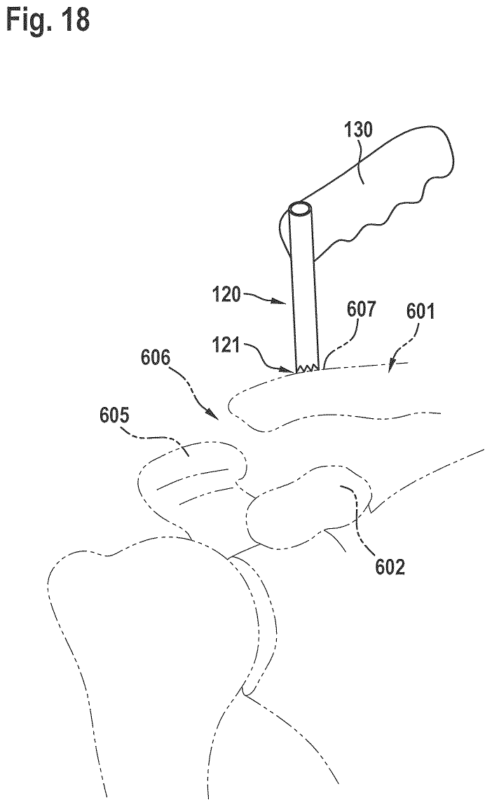

FIG. 1 shows a perspective view of the bone fixation device 100 for fixation of a first bone and a second bone to each other according to the first embodiment of the present invention. FIGS. 2 to 8 show instruments/tools that can be used in a bone fixation method for fixation of a first bone and a second bone to each other by using the bone fixation device 100 of FIG. 1. As will be described later with reference to FIGS. 9 to 17, the bone fixation device 100 can advantageously be used for bone fixation in a bone fracture, in particular a lesser trochanter fracture, in which a lesser trochanteric fragment is detached from the femur bone and, with reference to FIGS. 18 to 24, for bone fixation in a joint dislocation, in particular an acromioclavicular joint dislocation.

For fixation of the first bone and the second bone to each other, the first bone and the second bone need to be brought close to each other.

To this end, the bone fixation device 100 includes a first fixation element 1, a second fixation element 2 and a flexible strand 3. The first fixation element 1 is configured for passing through a hole of the first bone and a hole of the second bone and for engaging the second bone. The second fixation element 2 is configured for engaging the first bone and securing the flexible strand 3. The flexible strand 3 is used for connecting the first fixation element 1 and the second fixation element 2 to each other, in particular for pulling them towards each other, and thereby achieving fixation of the first bone and the second bone to each other.

As can be seen from FIG. 1, the first fixation element 1 includes, in particular is shaped as, a hollow cylinder 10. The hollow cylinder 10 extends in a direction of a longitudinal axis 405 from a first end 4 to a second end 5 and has a wall 11 defining a cylinder hollow space 15. In particular, the hollow cylinder 10 is a right circular hollow cylinder. The hollow cylinder 10 has an inner circumferential surface 18 that surrounds or defines the cylinder hollow space 15 and an outer circumferential surface 17.

An outer diameter 400 of the hollow cylinder 10 lies between 3 mm and 4 mm. A length 401 of the hollow cylinder 10 lies between 12 mm to 17 mm. A thickness 404 of the wall 11 of the hollow cylinder 10 lies between 0.5 mm and 1 mm.

Further, the first fixation element 1 is configured for receiving the flexible strand 3. For this purpose, the first fixation element 1 includes two openings 8. The openings 8 are in particular wall holes 80 formed in the wall 11 and configured for receiving the flexible strand 3.

The wall holes 80 are in particular through holes and communicate with the cylinder hollow space 15. The wall holes 80 extend in a direction of a thickness of the wall 11, in particular in a direction that is perpendicular to the wall 11, i.e., to the outer circumferential surface 17 and the inner circumferential surface 18. In other words, the wall holes 80 extend in a direction parallel to a normal vector of the wall of the first fixation element 1. It is, however, also possible that the wall holes 80 extend in a direction obliquely to the wall 11. The shape and the size of the wall holes 80 are chosen such that the flexible strand 3 can be passed through them.

It can further be seen from FIG. 1 that the wall holes 80 are arranged in a row parallel to a longitudinal axis 405 of the hollow cylinder 10 (first fixation element 1) and symmetrically to a central transverse axis 406 of the hollow cylinder 10 (first fixation element 1).

In FIG. 1, the flexible strand 3 is shown in a received state by the first fixation element 1. In other words, it is shown in a passed-through state through the wall holes 80. To this end, the flexible strand 3 may in particular be provided separately from the first fixation element 1 in the bone fixation device 100 and passed through the wall holes 80 during bone fixation performed by the surgeon before the first fixation element 1 is passed through the hole of the first bone and the hole of the second bone. Alternatively, the flexible strand 3 may already be passed through the wall holes 80 of the first fixation element 1 in the bone fixation device 100. In other words, the bone fixation device 100 may be prefabricated such that the first fixation element 1 is preloaded on the flexible stand 3/such that the flexible strand 3 is already passed through the wall holes 80 of the first fixation element 1, in particular fixed to the first fixation element 1. In the latter case, the relative position of the flexible strand 3 with respect to the first fixation element 1 is fixed.

When the flexible strand 3 is received by the first fixation element 1, three strand regions, namely a first strand region 31, a second strand region 32 and a third strand region 33, of the flexible strand 3 are advantageously defined.

The first strand region 31 extends from the one opening 8, in this case the one wall hole 80, in a direction away from the first fixation element 1. The second strand region 32 extends from the other opening 8, in this case the other wall hole 80, in a direction away from the first fixation element 1. The third strand region 33 connects the first strand region 31 to the second strand region 32. The third region 33 is a middle region between the first strand region 31 and the strand second region 32 and is arranged in the cylinder hollow space 15. The three strand regions 31, 32, 33 advantageously form a loop on the side of the cylinder hollow space 15 of the first fixation element 1. On the other side of the flexible strand 3, the flexible strand 3 includes a first strand end 34 and a second strand end 35 as free ends, when the flexible strand 3 is not secured to the second fixation element 2.

The second fixation element 2 is formed as a circular plate-shaped element. In particular, the second fixation element 2 may have a diameter 402 that lies between 10 mm and 15 mm. As can be viewed from FIG. 1, the second fixation element 2 includes two through holes 20 for receiving the flexible strand 3. As mentioned above, the flexible strand 3 is used for connecting the first fixation element 1 to the second fixation element 2 for fixing the first bone and the second bone to each other.

For this purpose, the first strand end 34 can particularly be passed through the first through hole 20 of the second fixation element 2 and the second strand end 35 through the second hole 20 of the second fixation element 2. Then, the first strand region 31 and the second strand region 32 or the two (free) strand ends 34, 35 can advantageously be tied together/to each other and/or around/to a region 21 of the second fixation element 2 between the through holes 20. In particular, the flexible strand 3 may advantageously be passed through the through holes 20 such that pulling the flexible strand 3 causes the second fixation element 2 move in a direction opposite to the pulling direction for engaging the second bone.

For facilitating a controlled advancement of the first fixation element 1 through the hole of the first bone and the second bone, the first fixation element 1 includes two proximal mating elements 16 for mating two distal mating elements 111 of a pusher 110 shown in FIG. 2. The pusher 110 is configured for pushing and in particular also for rotating the first fixation element 1 through the hole of the first bone and the second bone. Advantageously, the proximal mating elements 16 of the first fixation element 1 each have a complementary shape to the corresponding distal mating element 111 of the pusher 110. In particular, the proximal mating elements 16 of the first fixation element 1 can be identical to each other. Similarly, the distal mating elements 111 of the pusher 110 can be identical to each other.

In the present embodiment, the proximal mating elements 16 of the first fixation element 1 are formed as grooves. Accordingly, the distal mating elements 111 of the pusher 110 are formed as projections, in particular tongues, that are configured to mate with the grooves of the first fixation element 1.

For passing the first fixation element 1 through the hole of the first bone and the hole of the second hole, the surgeon places the pusher 110 onto the first fixation element 1 such the proximal mating elements 16 of the first fixation element 1 are mated with the distal mating elements 111 of the pusher 110. Then, the surgeon pushes and may in particular also rotate the pusher 110 so that the first fixation element 1 passes through the hole of the first bone and the hole of the second bone.

In order to further facilitate the insertion and the advancement of the first fixation element 1 through the hole of the first bone and the hole of the second bone, an outer edge 14 of a distal end face 13 of the first fixation element 1 is rounded.

For achieving the fixation of the first bone and the second bone with each other, the first fixation element 1 with the flexible strand 3 being passed through the wall holes 80 is pushed through the hole of the first bone and the hole of the second bone so that it exits the hole of the second bone or i.e., until it has come out of the hole of the second bone. When the first fixation element 1 has completely exited/come out of the hole of the second bone, the first fixation element 1 falls/rotates/pivots or, seen from the side of the surgeon, is let to fall/rotate/pivot, so that the first fixation element 1 can engage the second bone. To this end, the surgeon advantageously releases/reduces the tension in the flexible strand 3. It is understood that for this purpose the surgeon holds the first strand region 31 and the second strand region 32 while pushing the first fixation element 1 through the hole of the first bone and the hole of the second bone. In other words, the first strand region 31 and the second strand region 32 are located outside the hole of the first bone (and the hole of the second bone) while the first fixation element 1 is pushed through the hole of the first bone and the hole of the second bone.

A more detailed description of the bone fixation method by using the fixation device 100 according to a first embodiment and a second embodiment in two advantageous cases will be made with reference to FIGS. 9 to 17 and 18 to 24, respectively. Before that, the different instruments that can be during the bone fixation method will be described.

As already mentioned, FIG. 2 shows the pusher 110 to be used for pushing, and in particular also for rotating, the first fixation element 1 for advancing it through the hole of the first bone and the hole of the second bone.

The pusher 110 is formed as a hollow cylinder and has a pusher hollow space 112, in which the flexible strand 3 may be preloaded and kept in tension. The pusher 110 is provided with pusher distance marks 113, based on which the surgeon can check how deep inside the hole of the first bone and/or the hole of the second bone the first fixation element 1 has been pushed.

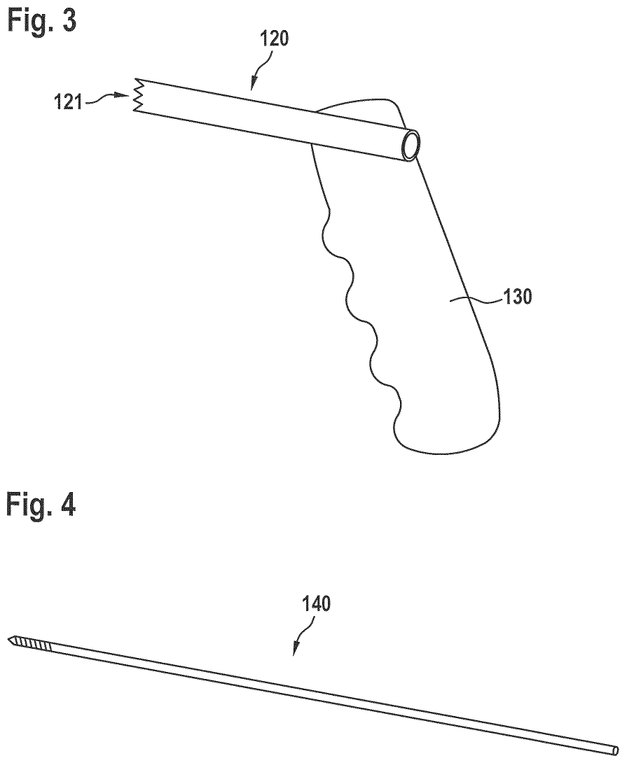

FIG. 3 shows a protective sleeve 120 formed as a hollow cylinder. The protective sleeve is in particular used for passing therethrough the first fixation element 1 and advancing it up to the bone hole. In order to facilitate a good grip and control over the protective sleeve, a handle 130 is attached to the protective sleeve 120. For achieving a more stable placement of the protective sleeve 120 against the first bone, the distal end of the protective sleeve 120 is provided with gripping means 121 that are configured to grip on the bone. In particular, the gripping means 121 may include a plurality of teeth.

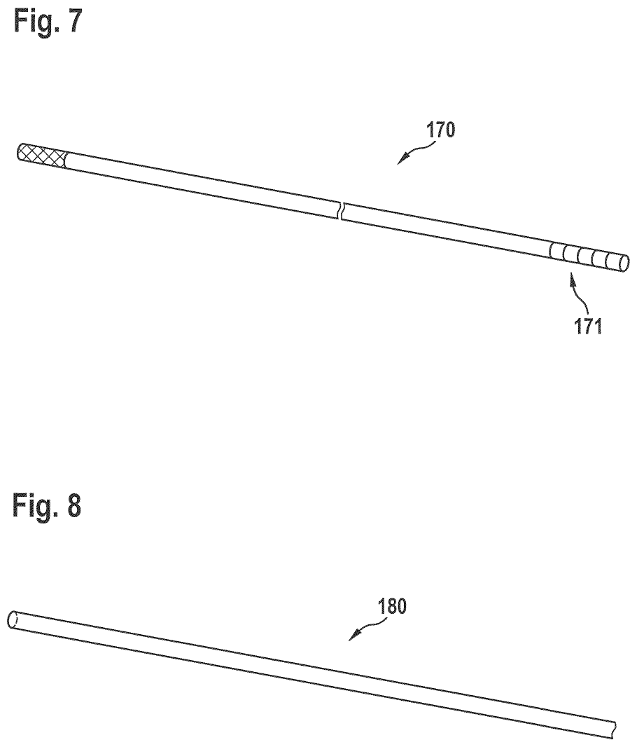

FIG. 4 shows a threaded Kirschner wire (K-wire) 140. The Kirschner wire 140 is configured to be passed through the first bone and the second bone and used for securing the second bone. By the advancement of the Kirschner wire 140 through the first bone and the second bone, a preliminary bone hole, in particular a preliminary hole of the first bone and a preliminary hole of the second bone are formed. Further, the Kirschner wire 140 can advantageously be used for passing a cannulated drill 170 (FIG. 7) over the Kirschner wire 140 for drilling the hole of the first bone and the hole of the second bone. The Kirschner wire 140 preferably has a diameter between 1.9 mm and 2.1 mm, in particular 2 mm.

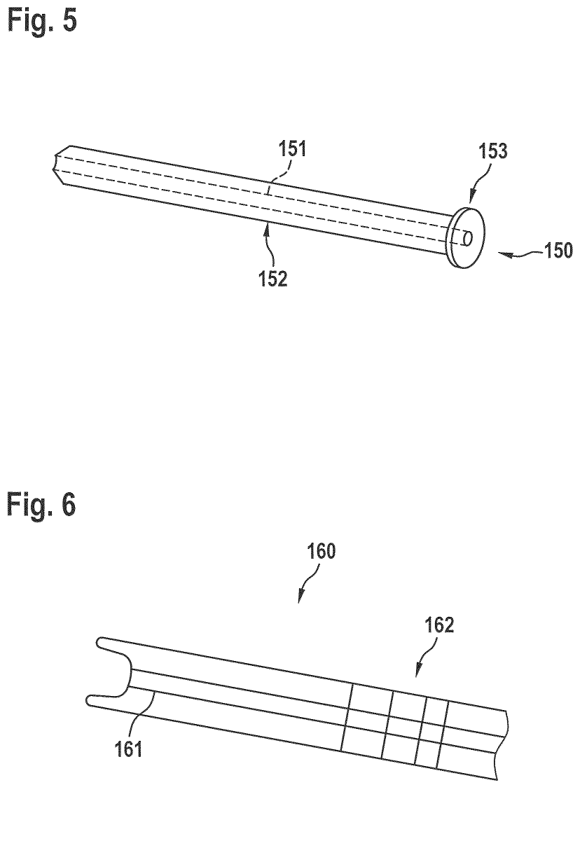

FIG. 5 shows a guide sleeve 150 for guiding the Kirschner wire 140, while the Kirschner wire 140 is being passed through the first bone and the second bone. To this end, the guide sleeve 150 preferably has a guide sleeve hollow space 151 for guiding the Kirschner wire 140 therethrough. The guide sleeve hollow space 151 is a longitudinal channel and preferably has a diameter between 1.9 mm and 2.1 mm, in particular 2 mm. Preferably, a shape and a size of the cross-section of the guide sleeve hollow space 151 are the same or substantially the same with a shape and a size of the cross-section of the Kirschner wire 140. The Kirschner wire 140 can be guided through the guide sleeve hollow space 151 in a sliding manner.

Further, the guide sleeve 150 is configured such that it is inserted in the protective sleeve 120. In particular, as can be seen from FIG. 5, the guide sleeve 150 includes a main sleeve body 152 that is insertable in the protective sleeve 120, and a stop means 153 for securing the position of the main sleeve body 152 relative to the protective sleeve 120 in an axial direction. The stop means 153 is configured such that the guide sleeve 150 abuts against a proximal end face of the protective sleeve 120 when the whole main region 152 is inserted in the protective sleeve 120. Preferably, the stop means 153 is formed as a flange.

FIG. 6 shows a ruler 160 that is used for deriving a length of the Kirschner wire 140 between the proximal cortex of the first bone and a distal end of the Kirschner wire 140. To this end, the ruler has a longitudinal ruler channel 161 to receive the Kirschner wire 140 and is provided with ruler distance marks 162.

FIG. 7 shows the cannulated drill 170 for drilling the bone hole. The cannulated design of the drill 170 has the advantage that it can be passed over the Kirschner wire 140, the latter acting as a guiding element for the cannulated drill 170. Further, the drill 170 is configured such that it can be inserted in the protective sleeve 120.

In addition, the drill 170 is provided with drill distance marks 171 that help the surgeon know how deep the drill 170 has been advanced inside the first bone and the second bone, and consequently the depth of the drilled bone hole at any time during the procedure. Thus, the surgeon can accurately drill the needed hole through the first bone and the second bone.

FIG. 8 shows a guiding element 180 in the form of a guidewire with a rounded tip. The guiding element 180 is configured for guiding the first fixation element 1 through the hole of the first bone and the hole of the second bone. To this end, the first fixation element 1 is passed over the guiding element 180 so that it can be advanced through the hole of the first bone and the hole of the second bone by the pusher 110 described above.

FIGS. 9 to 17 show steps of a bone fixation method for bone fixation in a lesser trochanter fracture according to a first embodiment by using the previously described bone fixation device 100 of FIG. 1 and the instruments of FIGS. 2 to 8.

As can be seen from FIG. 9, in the lesser trochanter fracture, a lesser trochanteric fragment 501 is detached from the femur bone 501. The femur bone 501 corresponds to the first bone and the lesser trochanteric fragment 502 to the second bone within the context of the present invention. For treating the lesser trochanter fracture, the lesser trochanteric fragment 502 particularly needs to be reduced and fixed to the femur bone 501.

Here, the lesser trochanter fracture is presented as a component of another type of hip fracture, in particular an intertrochanteric fracture. For treating the intertrochanteric fracture, an intramedullary nail 508 is provided inside the femur bone 501. The bone fixation method may however also be applied to an isolated lesser trochanter fracture, i.e. to a lesser trochanter fracture that has occurred without any other type of hip fracture.

The femur bone 501 can be a human femur bone of a patient. However, the femur bone 501 can also be a human cadaveric femur bone or a surgical phantom including or representing a human cadaveric femur bone for training purposes.

After the surgeon has gained access to the femur bone 501, the surgeon places, in a first step of the bone fixation method shown in FIG. 9, the protective sleeve 120 against the lateral cortex 505 of the femur bone 501 (proximal cortex of the first bone) so that the gripping means 121 of the protective sleeve 120 grip on the lateral cortex 505 and the position of the protective sleeve 120 is secured.

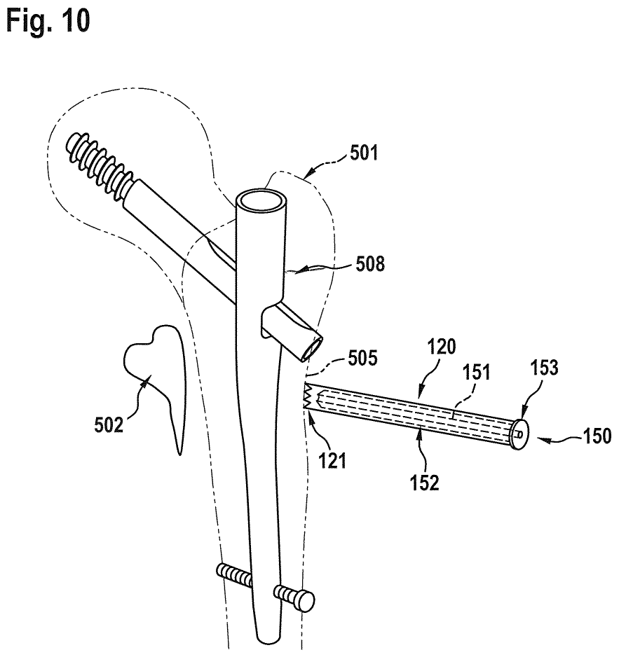

In a second step shown in FIG. 10, the guide sleeve 150 is inserted in the protective sleeve 120 while holding the latter in place. In particular, the main sleeve body 152 of the guide sleeve 150 is inserted in the protective sleeve 120 until the stop means 153 of the guide sleeve 150 abuts against the protective sleeve 120, in particular a proximal end face of the protective sleeve 120. A relative position of the guide sleeve 150 in an axial direction with respect to the protective sleeve 120 can thereby be secured.

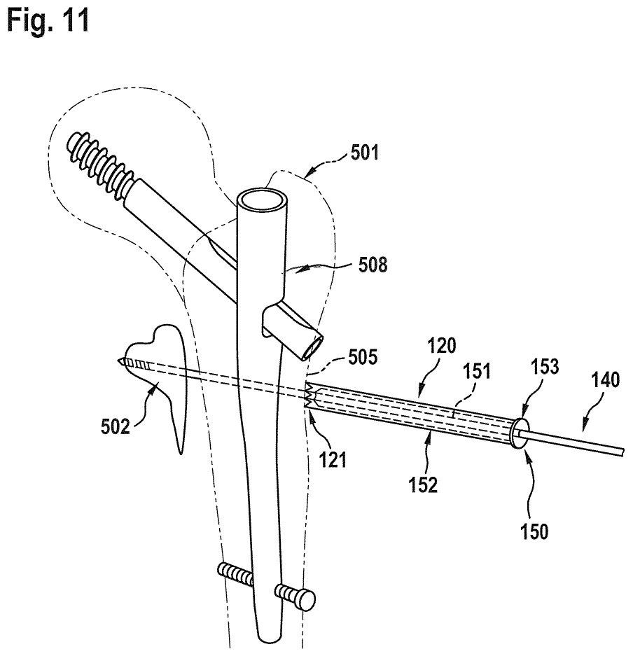

In a third step shown in FIG. 11, the threaded Kirschner wire 140 is inserted in the guide sleeve hollow space 151 (formed as a longitudinal channel) and pushed through the femur bone 501 and the lesser trochanteric fragment 502. This results in the formation of a preliminary femur bone hole (preliminary hole of the first bone bone) and a preliminary lesser trochanteric fragment hole (preliminary hole of the second bone bone) and in securing the lesser trochanteric fragment 502.

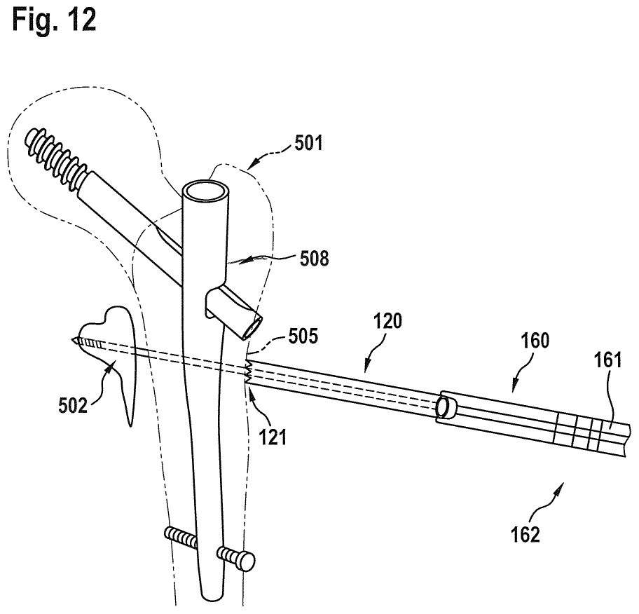

In order to find out how deep the Kirschner wire 140 has been inserted, the previously described ruler 160 is advantageously used in a fourth step shown in FIG. 12.

The surgeon places, in particular slides, the ruler 140 over the Kirschner wire 140 so that the Kirschner wire 140 is received in the longitudinal ruler channel 161, and advances the ruler 160 until the ruler 160 has reached the proximal end of the guide sleeve 150.

Thereby, a distance between a proximal end of the guide sleeve 150 and a proximal end of the Kirschner wire 140 can be measured. In other words, a distance of the Kirschner wire 140 extending outwards from the guide sleeve 150 can be measured. Thus, as the overall length of the Kirschner wire 140 and the length of the guide sleeve 150 are known and the distance between the proximal end of the guide sleeve 150 and the proximal end of the Kirschner wire 140 is measured by the ruler 160, the distance between the lateral cortex 505 of the femur bone 501 and the distal end of the Kirschner wire 140, namely the insertion depth of the Kirschner wire 140, can be derived. The insertion depth is indicative of the position of the lesser trochanteric fragment 502.

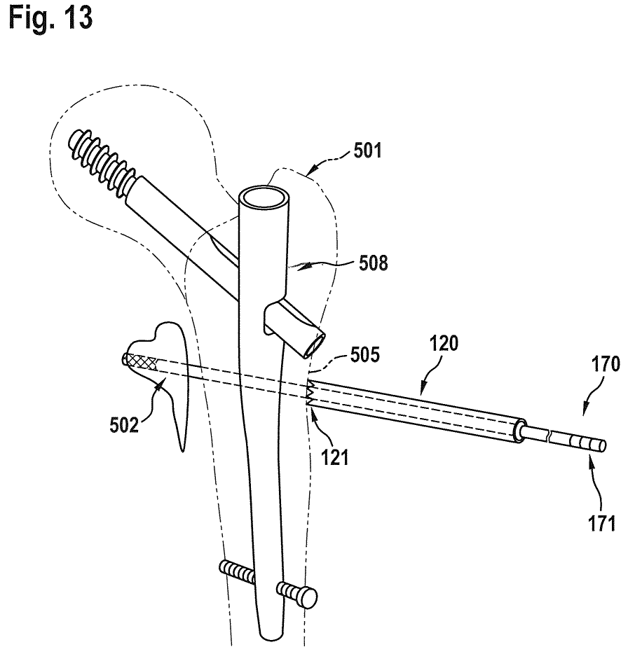

Based on the derived insertion depth of the Kirschner wire 140, a femur bone hole 503 (hole of the first bone) and a lesser trochanteric fragment hole 504 (hole of the second bone) are drilled in a fifth step shown in FIG. 13. To this end, the cannulated drill 170 is guided over the Kirschner wire 140 until the bone hole 502 is drilled. It is noted that the femur bone hole 503 corresponds to the hole of the first bone or also called first-bone hole, wherein the lesser trochanteric fragment hole 504 corresponds to the hole of the second bone or also called second-bone hole.

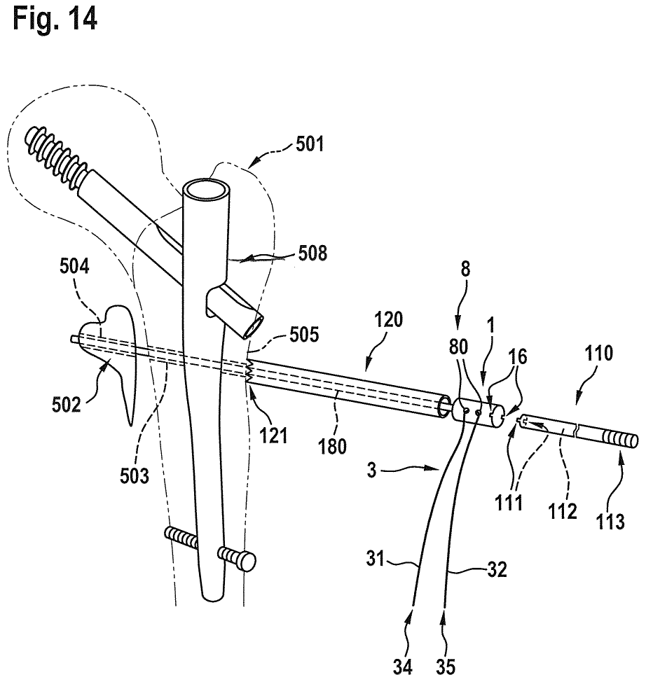

Then, in a sixth step, the Kirschner wire 140 is removed while the cannulated drill 170 is maintained in place (without drilling) and the guiding element 180 is inserted through the cannulated drill 170 so that a distal end of the guiding element (guidewire) 180 exits the lesser trochanteric fragment hole 504. When the guiding element 180 has reached the desired position, the cannulated drill 170 is removed. It is understood that the cannulated drill 170 is itself used here as a guide for inserting the guiding element 180 in the femur bone hole 503 and the lesser trochanteric fragment hole 504. FIG. 14 shows the state where the cannulated drill 170 has been removed and the guiding element 180 is inserted in the femur bone hole 503 and the lesser trochanteric fragment hole 504.

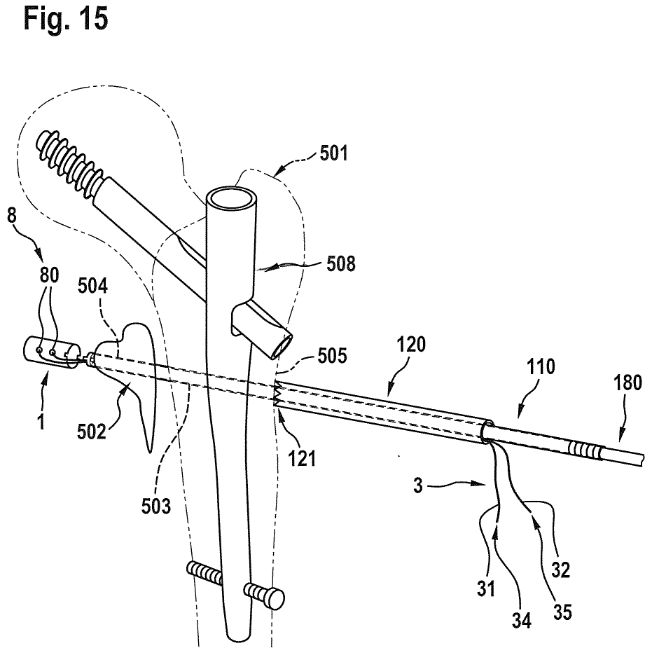

Further with reference to FIGS. 14 and 15, in a seventh step, the first fixation element 1 is pushed by the pusher 110 through the femur bone hole 503 and the lesser trochanteric fragment hole 504. To this end, the first fixation element 1 and the pusher 110 are placed over the guiding element 180. The flexible strand 3, in particular the first strand end 34 and the second strand end 35, is/are passed through the pusher hollow space 151. The surgeon mates the pusher 110 with the first fixation element 1 via the distal mating elements 111 of the pusher 110 and the proximal mating elements 16 of the first fixation element 1. The flexible strand 3 is preloaded and kept in tension in the pusher hollow space 151. While the flexible strand 3 is in this state, the surgeon pushes the pusher 110 until the first fixation element 1 exits the lesser trochanteric fragment hole 504. This is shown in FIG. 15.

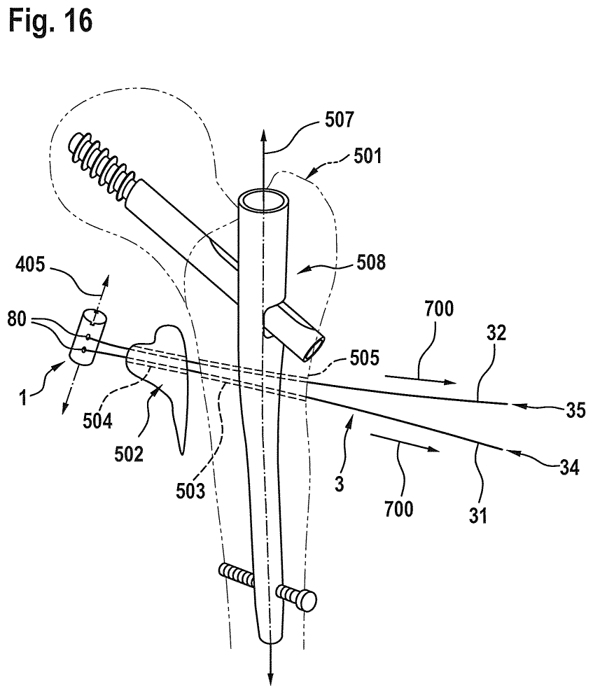

Then, the surgeon removes the pusher 110 out of the lesser trochanteric fragment 502 and releases the flexible strand 3 from the pusher 110. Thus, the tension in the flexible strand 3 is released. This results in the first fixation element 1 rotating/falling/pivoting so that the first fixation element 1 may be arranged with its longitudinal axis 405 being parallel or substantially parallel to a longitudinal axis 507 of the femur bone 501. This state is shown in FIG. 16.

With further reference to FIG. 16, in an eighth step, the first strand region 31 and the second strand region 32 of the flexible strand 3 are pulled so that the first fixation element 1 is pulled towards the lesser trochanteric fragment 502 (pulling direction indicated by arrows 700). This results in the first fixation element 1 abutting against the medial end of the lesser trochanteric fragment 502 and thereby the lesser trochanteric fragment 502 being reduced.

In a ninth step, the first strand end 34 of the flexible strand 4 may be passed through the one hole 20 of the second fixation element 2 and the second strand end 35 through the other hole 20 of the second fixation element 2. The second fixation element 2 is placed on the lateral cortex of the femur bone 501, and the first strand region 31 and the second strand region 32 of the flexible strand 3 are secured to the second fixation element 2 so that the lesser trochanteric fragment 502 is fixed to the femur bone 501.

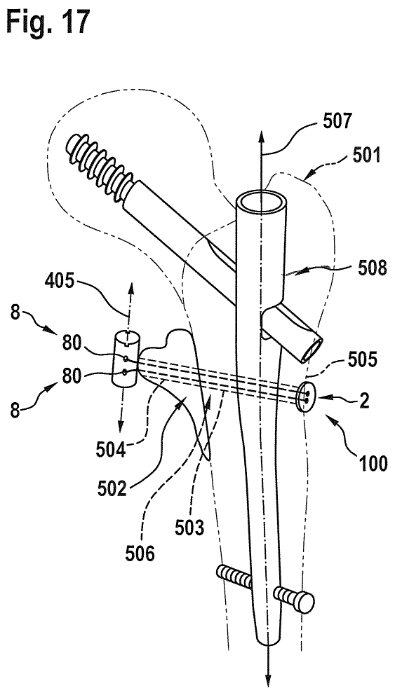

FIG. 17 shows the reduced and fixed state of the lesser trochanteric fragment 502 and thus the fixed state of the femur bone 501 and the lesser trochanteric fragment 502 to each other. In this state, the flexible strand 3 forms a closed loop. Further, it can be seen from FIG. 17 that the first fixation element 1 is arranged on, i.e. engages the femur bone 501, in particular its medial cortex, such that the longitudinal axis 405 of the first fixation element 1 may be parallel or at least substantially parallel to the longitudinal axis 507 of the femur bone 501.

FIGS. 18 to 24 show phases/stages of a bone fixation method according to a second embodiment for bone fixation in an acromioclavicular joint dislocation by using the previously described bone fixation device 100 of FIG. 1 and the instruments of FIGS. 2 to 8.

As can be seen from FIG. 18, in the case of an acromioclavicular joint dislocation, i.e. a dislocation of the acromioclavicular joint 606, the outer end of the clavicle 601 is dislocated from the acromion 605. In order to repair the dislocation, the acromioclavicular joint 606 needs to be reduced. To this end, the clavicle 601 and the coracoid process 602 can be fixed to each other by using the bone fixation device 100 of FIG. 1. Here, the clavicle 601 corresponds to the first bone and the coracoid process 602 to the second bone within the context of the present invention. The clavicle 601 and the coracoid process 602 can be of a patient. However, they can also be human cadaveric bones or part of a surgical phantom for training purposes.

After the surgeon has gained access to the clavicle 601, the surgeon places, as shown in FIG. 18, the protective sleeve 120 against the cranial cortex 607 of the clavicle 601 (proximal cortex of the first bone) so that the gripping means 121 of the protective sleeve 120 grip on the cranial cortex 607 and the position of the protective sleeve 120 is secured.

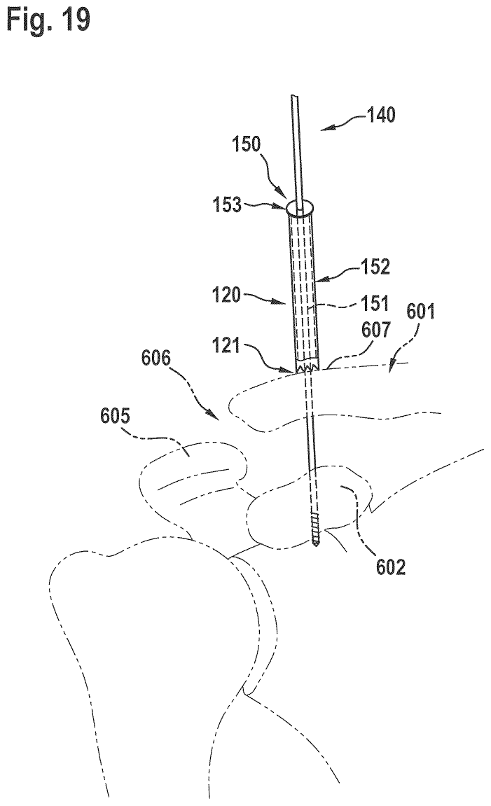

Then, the surgeon inserts the guide sleeve 150 in the protective sleeve 120 while holding the latter in place. In particular, the main sleeve body 152 of the guide sleeve 150 is inserted in the protective sleeve 120 until the stop means 153 of the guide sleeve 150 abuts against the protective sleeve 120, in particular a proximal end face of the protective sleeve 120. A relative position of the guide sleeve 150 in an axial direction with respect to the protective sleeve 120 can thereby be secured. In a further step, the threaded Kirschner wire 140 is inserted in the guide sleeve hollow space 151 and pushed through the clavicle 601 and the coracoid process 602. This results in the formation of a preliminary clavicular bone hole (preliminary hole of the first bone bone) and a preliminary coracoid process hole (preliminary hole of the second bone bone) and in securing the coracoid process 602. This phase of the bone fixation method is shown in FIG. 19.

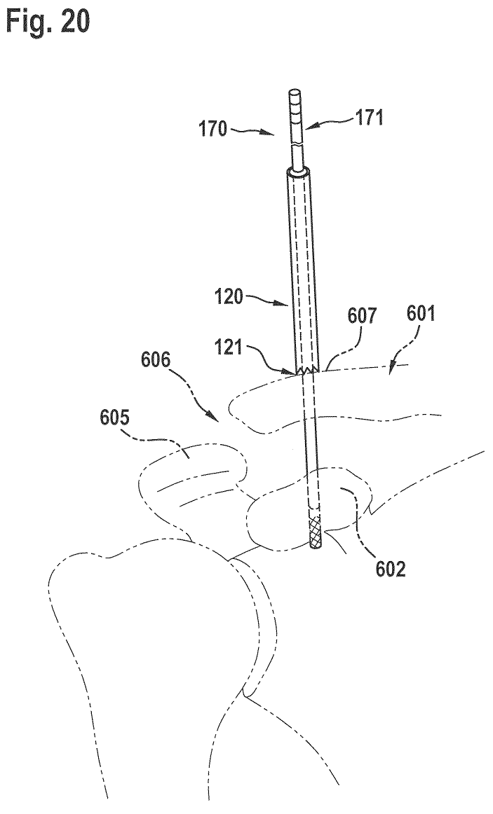

Then, a clavicular hole 603 and a coracoid process hole 604 are drilled as shown in FIG. 20. To this end, the cannulated drill 170 is guided over the Kirschner wire 140 until the clavicular hole 603 and the coracoid process hole 604 are drilled. It is noted that the clavicular hole 603 corresponds to the hole of the first bone or also called first-bone hole, wherein the coracoid process hole 604 corresponds to the hole of the second bone or also called second-bone hole. For drilling the clavicular hole 603 and the coracoid process hole 604, the insertion depth of the Kirschner wire 140 can be previously measured. For this purpose, the ruler 160 can be used. The surgeon places, in particular slides, the ruler 140 over the Kirschner wire 140 so that the Kirschner wire 140 is received in the longitudinal ruler channel 161, and advances the ruler 160 until the ruler 160 has reached the proximal end of the guide sleeve 150. Thereby, a distance between a proximal end of the guide sleeve 150 and a proximal end of the Kirschner wire 140 can be measured. Thus, as the overall length of the Kirschner wire 140 and the length of the guide sleeve 150 are known and the distance between the proximal end of the guide sleeve 150 and the proximal end of the Kirschner wire 140 is measured by the ruler 160, the distance between the cranial cortex 607 of the clavicle 601 and the distal end of the Kirschner wire 140, namely the insertion depth of the Kirschner wire 140, can be derived.

Then, the Kirschner wire 140 is removed while the cannulated drill 170 is maintained in place (without drilling) and the guiding element (guidewire) 180 is inserted through the cannulated drill 170 so that a distal end of the guiding element 180 exits the coracoid process hole 604. When the guiding element 180 has reached the desired position, the cannulated drill 170 is removed. FIG. 21 shows the state where the cannulated drill 170 has been removed and the guiding element 180 is inserted in the clavicular hole 603 and the coracoid process hole 604.

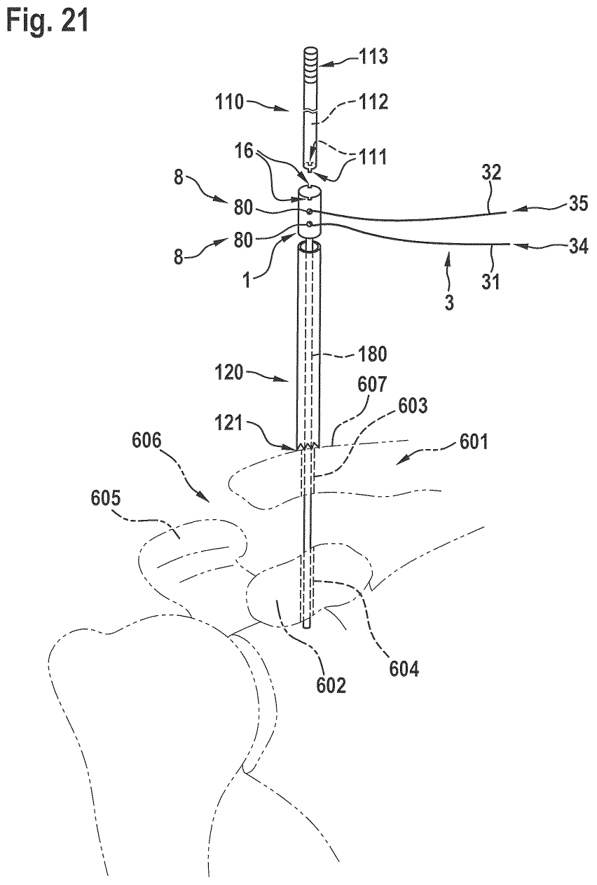

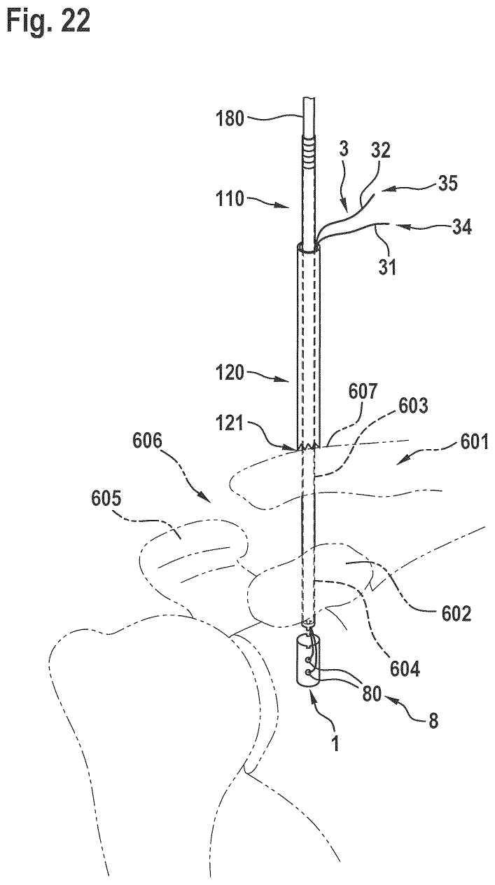

Further with reference to FIGS. 21 and 22, the first fixation element 1 is pushed by the pusher 110 through the clavicular hole 603 and the coracoid process hole 604. To this end, the first fixation element 1 and the pusher 110 are placed over the guiding element 180. The flexible strand 3, in particular the first strand end 34 and the second strand end 35, is/are passed through the pusher hollow space 151. The surgeon mates the pusher 110 with the first fixation element 1 via the distal mating elements 111 of the pusher 110 and the proximal mating elements 16 of the first fixation element 1. The flexible strand 3 is preloaded and kept in tension in the pusher hollow space 151. While the flexible strand 3 is in this state, the surgeon pushes the pusher 110 until the first fixation element 1 exits the coracoid process hole 604. This is shown in FIG. 22.

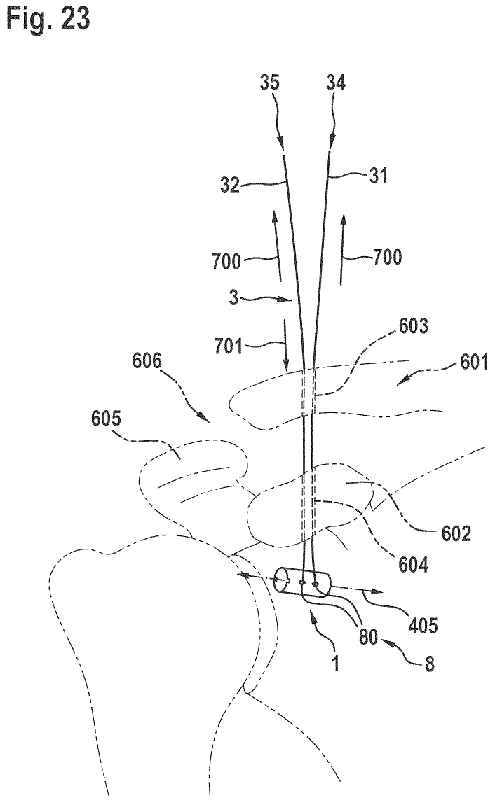

Then, the surgeon removes the pusher 110 out of the coracoid process 602 and releases the flexible strand 3 from the pusher 110. Thus, the tension in the flexible strand 3 is released. This results in the first fixation element 1 rotating/falling/pivoting so that the first fixation element 1 may be arranged with its longitudinal axis 405 being parallel or substantially parallel to a longitudinal axis of the coracoid process 602. This state is shown in FIG. 23.

With further reference to FIG. 23, the first strand region 31 and the second strand region 32 of the flexible strand 3 are pulled so that the first fixation element 1 is pulled towards the coracoid process 602 (pulling direction indicated by arrows 700). This results in the first fixation element 1 abutting against the caudal end of the coracoid process 602 and thereby engaging the coracoid process 602. Further, the clavicle 601 may be pushed in the opposite direction as indicated by the arrows 701 in FIG. 22 so that the clavicle 601 and the coracoid process 602 are brought (even) closer to each other.

Then, the first strand end 31 of the flexible strand 3 may be passed through the one hole 20 of the second fixation element 2 and the second strand end 35 through the other hole 20 of the second fixation element 2. The second fixation element 2 is placed on the cranial cortex of the clavicle 601, and the first strand region 31 and the second strand region 32 of the flexible strand 3 are secured to the second fixation element 2 so that the clavicle 601 and the coracoid process 602 are fixed to each other.

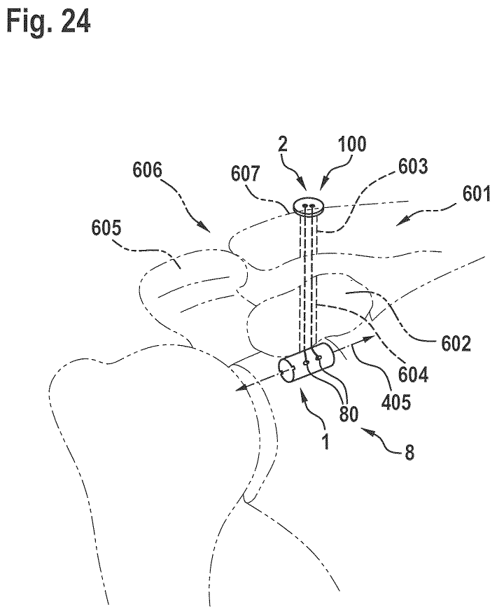

The fixed state of the clavicle 601 and the coracoid process 602 to each other is shown in FIG. 24. Due to the bone fixation of the bones by means of the bone fixation device 100, the acromioclavicular joint 606 is reduced and thus the acromioclavicular joint dislocation is overcome.

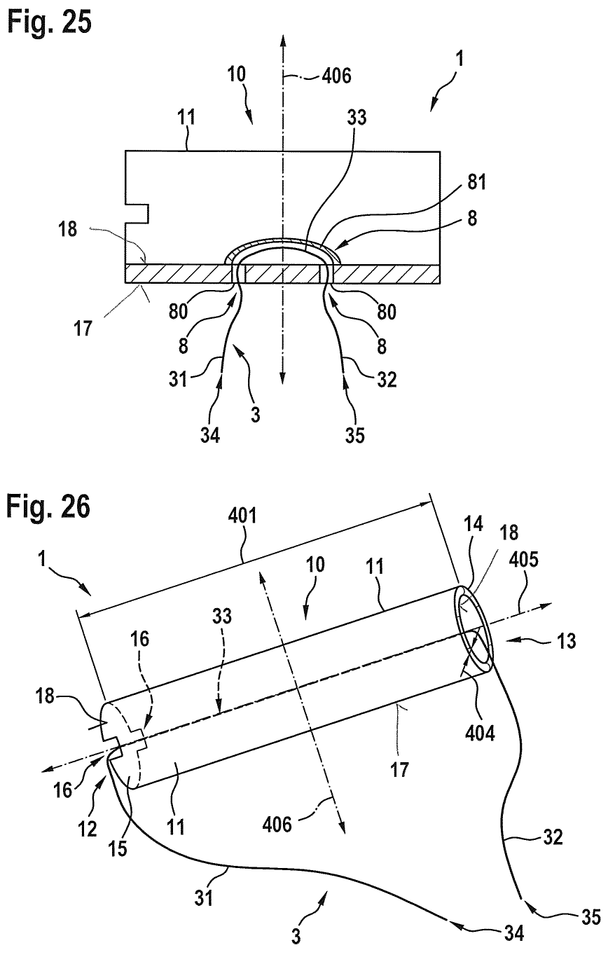

FIG. 25 shows a front cross-sectional view of a portion of a bone fixation device 100 according to a second embodiment for bone fixation.

Compared to the first fixation element 1 of the bone fixation device 100 according to the first embodiment, the first fixation element 1 of the bone fixation device 100 according to the second embodiment further includes an opening 8 formed as a pocket 81. The pocket 81 communicates with both wall holes 80 of the first fixation element 1 and is formed on the inner circumferential surface 18 of the hollow cylinder 10.

Thus, the flexible strand 3 can be passed through the pocket 81 by inserting it via one of the wall holes 80 and taking it out of the other wall hole 80. This means that part of the flexible strand 3 is positioned inside the pocket 81, and not inside the cylinder hollow space 15 of the first fixation element 1 as is the case with the first fixation element 1 of the bone fixation device 100 of the first embodiment.

Thus, the procedure of passing through the first fixation element 1 through the bone hole can be simplified for the surgeon, as the flexible strand 3 is prevented, without any specific action required by the surgeon, from intervening with the guiding element 180 while guiding the first fixation element over the guiding element 180.

The described bone fixation method has the advantage that the first bone and the second bone can be fixed to each other in a minimal invasive, stable, precise and accurate way. The steps that the surgeon has to follow can be easily trained and are intuitive so that any potential mistakes during the procedure can be minimized or eliminated.

FIG. 26 shows a perspective view of a portion of a bone fixation device 100 according to a third embodiment.

In the bone fixation device 100 according to third embodiment, the walls holes 80 are omitted and the cylinder hollow space 15 is used for receiving the flexible strand 3. Thus, the cylinder hollow space 15 can be considered as an opening (but not formed in the wall 11) that is configured to receive the flexible strand 3. Before inserting the first fixation element 1 through the hole of the first bone and the hole of the second bone, the flexible strand 3 is passed through the hollow space 15. To this end, the flexible strand 3 is passed through the first end 4 and the second end 5 of the hollow cylinder 10.

By using the cylinder hollow space 15 for passing through the flexible strand 3 and omitting the wall holes 80, the design of the bone fixation device 100 may be simplified. On the other hand, the first fixation element 1 may still include the wall holes 80, while the surgeon may choose whether to pass the flexible strand 3 through the wall holes 80 or the first end 4 and the second end 5 of the hollow cylinder 10. An advantage of having the wall holes 80 and using them for receiving the flexible strand 3 is that the wall holes 80 can be formed as close as possible to the middle portion of hollow cylinder 10 in the direction of the longitudinal axis 405. Thus, a distance between the region of the hollow cylinder 10 from which the flexible strand 3 exits the hollow cylinder 10 and the hole of the second bone can be reduced. Thereby, bending of the flexible strand 3 may be reduced. In particular, positioning of the wall holes 80 can be chosen such that a contact of the flexible stand 3 at the edge of the hole of the second bone can be prevented. In particular, the smallest distance between the wall holes 80 may be smaller than the diameter of the hole of the first bone.

The rest of the design of the first fixation element 1 according to the third embodiment corresponds to that of the first fixation element 1 of the first embodiment.

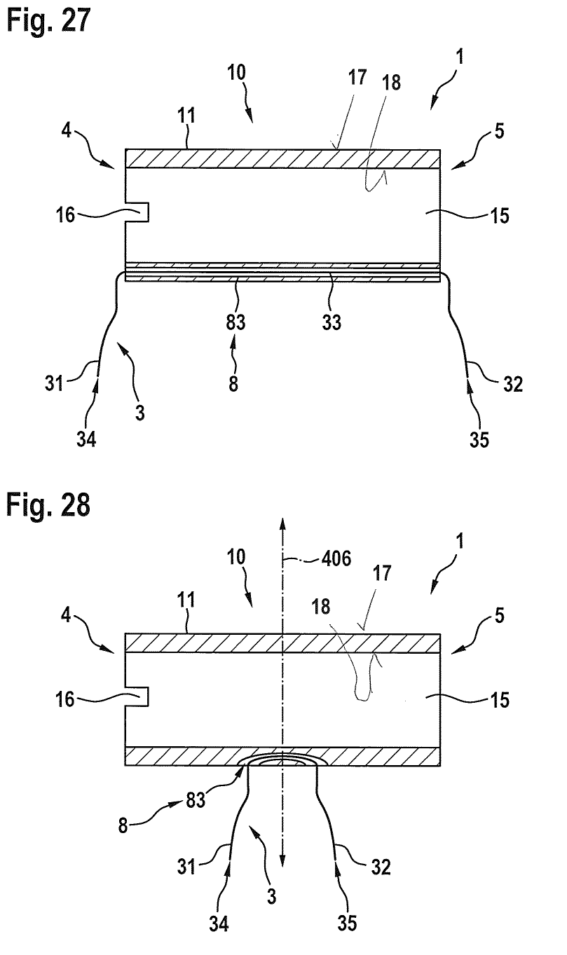

FIG. 27 shows a front cross-sectional view of a portion of a bone fixation device 100 according to a fourth embodiment.

The first fixation element 1 of the bone fixation device 100 according to the fourth embodiment includes an opening 8 formed as a channel 83 inside the wall 11. The channel 83 extends from the first end 4 of the hollow cylinder 10 to the second end 5 of the hollow cylinder 10 parallel to the longitudinal axis 405.

The design of the first fixation element 1 according to the fourth embodiment has the advantage that the flexible strand 3 can be received by the first fixation element 1 without being positioned inside the cylinder hollow space 15.

FIG. 28 shows a front cross-sectional view of a portion of a bone fixation device 100 according to a fifth embodiment.

The first fixation element 1 of the bone fixation device 100 according to the fifth embodiment includes an opening 8 formed as a channel 83. The channel 83 is formed inside the wall 11 and starts from the outer circumferential surface 17 of the hollow cylinder 10 and also ends at the outer circumferential surface 17 of the hollow cylinder 10. The channel 83 is in particular formed as a curved channel.

The flexible strand 3 can thus be passed through the channel 83. Thus, the flexible strand 3 can be received by the first fixation element 1 without being positioned inside the cylinder hollow space 15.

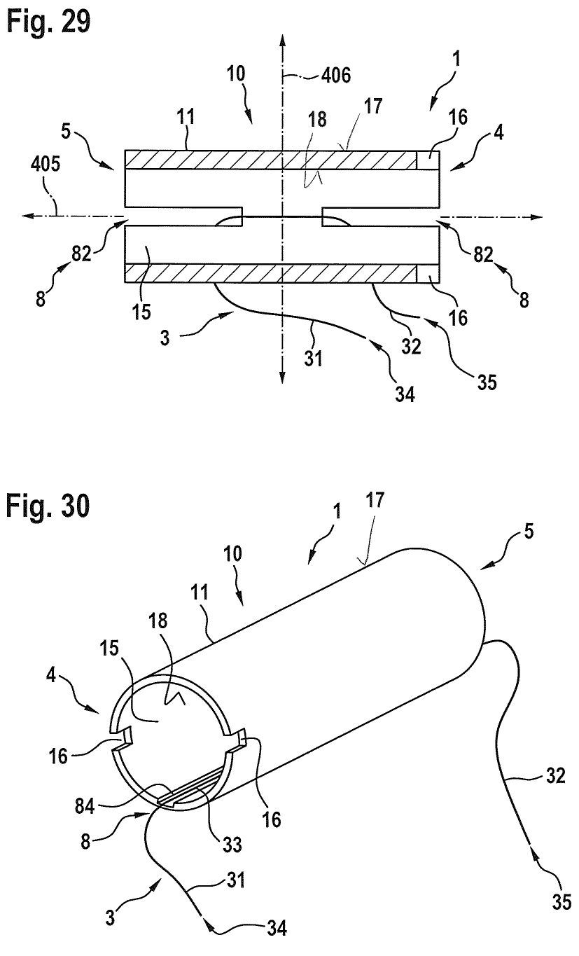

FIG. 29 shows a back cross-sectional view of a portion of a bone fixation device 100 according to a sixth embodiment.

The first fixation element 1 of the bone fixation device 100 according to the sixth embodiment includes two openings 8 formed as slits 82 in the wall 11.

One of the slits 82 extends from the first end 4 of the hollow cylinder 10 in the direction from the first end 4 to the second end 5 of the hollow cylinder 10. In particular, the slit 82 extends from the first end 4 of the hollow cylinder 10 to a position within a middle region of the hollow cylinder 10 between the first end 4 and the second end 5.

The other slit 82 extends from the second end 5 of the hollow cylinder 10 in the direction from the second end 5 to the first end 4 of the hollow cylinder 10. In particular, the other slit 82 extends from the second end 5 of the hollow cylinder 10 to a position within the middle region of the hollow cylinder 10 between the first end 4 and the second end 5.

As can be further seen from FIG. 21, the slits 82 are aligned with each other in the circumferential direction. This means that the longitudinal axes of the slits 82 are collinear. It can further be seen that the slits 82 extend parallel to the longitudinal axis 405 of the hollow cylinder 10.

The flexible strand 3 is passed through the slits 82 so that the flexible strand 3 is received by the first fixation element 1. In FIG. 21, the flexible strand 3 is shown in a received state.

FIG. 30 shows a perspective view of a portion of a bone fixation device 100 according to a seventh embodiment.

The first fixation element 1 of the bone fixation device 100 according to the seventh embodiment includes an opening 8 formed as a groove 84 formed in the inner circumferential surface 18 of the hollow cylinder 10. The groove 84 extends from the first end 4 to the second end 5 of the hollow cylinder 10, i.e. starts at the first end 4 and ends at the second end 5 of the hollow cylinder 10, parallel to the longitudinal axis 405.

The flexible strand 3 can thus be received in the groove 84, thereby reducing the risk of interference of the flexible strand 3 with the guiding element 180 used for guiding the first fixation element 1 and thus simplifying the procedure for the surgeon.

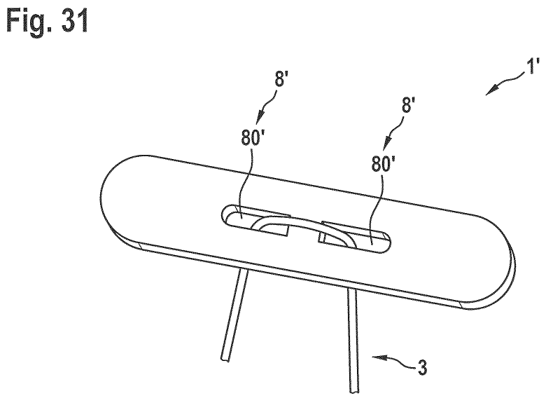

FIG. 31 shows a portion of a bone fixation device 100 for bone fixation in a lesser trochanter fracture that can be used in a bone fixation method according to a third embodiment.

Here, the used bone fixation device 100 differs from the previously described bone fixation devices 100 in that the first fixation element 1′ shown in FIG. 31 is formed as an elongated button, i.e. an elongated plate-shaped element with through holes 80′ (openings 8′), and not as a hollow cylinder. The through holes 80′ extend through the thickness of the first fixation element 1′.

The bone fixation method according to the third embodiment of the present invention preferably shares the first to fifth step of the bone fixation method according to the first embodiment. After the fifth step, the Kirschner wire 140 and the cannulated drill 170 are removed in the bone fixation method according to the present (third) embodiment. The first fixation element 1′ is then passed, in particular pushed, through the femur bone hole 503 and the lesser trochanteric fragment hole 504. To this end, the previously described pusher 110 can be used. However, as the first fixation element 1′ according to this embodiment does not have any proximal mating elements, the pusher 110 can be provided without its distal mating elements 111.

The flexible strand 3 can be preloaded and kept in tension in the pusher hollow space 151. While the flexible strand 3 is in this state, the surgeon pushes the pusher 110 until the first fixation element 1′ exits the lesser trochanteric fragment hole 504. Then, the surgeon releases the flexible strand 3 from the pusher 110. Thus, the tension in the flexible strand 3 is released. This results in the first fixation element 1′ rotating/falling/pivoting similarly to the first fixation element 1 of the previous embodiments.

It is noted that the first fixation element 1 according to the present invention may include a plurality of openings 8 that can be a combination of the openings 8 of the above described first fixation devices 100. For example, the first fixation element 1 may include two wall holes 80 and/or a pocket 81 and/or two slits 82 and/or a channel 83 and/or a groove 84 as the ones described above.

It is further noted that the bone fixation devices 100 according to the third, fourth, fifth, sixth and seventh embodiment can each be used in the bone fixation method as previously described with reference to FIGS. 9 to 17 and 18 to 24. Further, it is noted that the bone fixation device 100 of the present invention can be provided together with the pusher 110 as/in a kit. The kit may also include any or all of the rest of the instruments used in the previously described bone fixation method.

In addition to the foregoing description of the present invention, for an additional disclosure explicit reference is taken to the graphic representation of FIGS. 1 to 31.

Having thus described the invention of the present application in detail and by reference to embodiments thereof, it will be apparent that modifications and variations are possible without departing from the scope of the invention defined in the appended claims as follows:

1. A bone fixation device for fixation of a first bone and a second bone to each other in a bone fracture and/or a joint dislocation, including:
a first fixation element configured for passing through a hole of the first bone and a hole of the second bone and for engaging the second bone,
a flexible strand, and
a second fixation element configured for engaging the first bone and for securing the flexible strand,
wherein the first fixation element:
includes a hollow cylinder, wherein the hollow cylinder extends in a direction of a longitudinal axis from a first end to a second end and has a wall defining a cylinder hollow space and,
is configured for receiving the flexible strand.

2. The bone fixation device according to claim 1, wherein the first fixation element further includes at least one opening that is configured for receiving the flexible strand, in particular formed in and/or on the wall of the hollow cylinder.

3. The bone fixation device according to claim 2, wherein the at least one opening includes a wall hole formed in the wall of the hollow cylinder, in particular wherein the wall hole extends in a direction that is perpendicular or oblique to an outer circumferential surface and/or to an inner circumferential surface of the hollow cylinder.

4. The bone fixation device according to claim 3, wherein the wall hole is a through hole and communicates with the cylinder hollow space.

5. The bone fixation device according to claim 3, wherein the at least one opening includes a further wall hole, wherein the wall holes are arranged in a row in a direction from the first end to the second end, in particular parallel to the longitudinal axis, and/or arranged symmetrically to a central transverse axis of the hollow cylinder.

6. The bone fixation device according to claim 2, wherein the at least one opening includes a slit in the wall, the slit extending from the first end in the direction from the first end to the second end, in particular wherein the at least one opening includes a further slit in the wall, the further slit extending from the second end of the hollow cylinder in the direction from the second end to the first end.

7. The bone fixation device according to claim 2, wherein the at least one opening includes a channel formed inside the wall, the channel extending from the first end to the second end of the hollow cylinder.

8. The bone fixation device according to claim 2, wherein the at least one opening includes a groove formed in an inner circumferential surface of the hollow cylinder and extending in a direction from the first end to the second end, in particular from the first end to the second end.

9. The bone fixation device according to claim 2, wherein the at least one opening includes a pocket formed on an inner circumferential surface of the hollow cylinder and/or wherein the at least one opening includes a channel that extends through the wall of the hollow cylinder and starts from and ends at the outer circumferential surface of the hollow cylinder.

10. The bone fixation device according to claim 1, wherein an outer diameter of the hollow cylinder is equal to or smaller than 4 mm, preferably lies between 3 mm and 4 mm, and/or wherein a length of the hollow cylinder lies between 12 mm to 17 mm and/or wherein a thickness of the wall of the hollow cylinder lies between 0.5 mm and 1 mm and/or wherein an outer edge of a distal end face of the first fixation element is rounded.

11. The bone fixation device according to claim 1, wherein the first fixation element includes at least one mating element for mating with at least one mating element of a pusher for pushing and in particular also for rotating the first fixation element through the first bone and the second bone, preferably wherein the at least one mating element of the first fixation element includes a projection and/or a groove.

12. The bone fixation device according to claim 1, wherein the flexible strand is received by the first fixation element.

13. The bone fixation device according to claim 1, wherein the second fixation element is formed as a plate-shaped element and/or includes at least one hole for receiving the flexible strand and/or wherein the second fixation element is loaded on the flexible strand such and/or the flexible strand is configured such that pulling the flexible strand when received by the first fixation element and the second fixation element causes the second fixation element to move in a direction opposite to the pulling direction for engaging the first bone.

14. The bone fixation device according to claim 1, being configured for bone fixation in a lesser trochanteric fracture, in which a lesser trochanteric fragment is detached from the femur bone, wherein the first bone is the femur bone, and the second bone is the lesser trochanteric fragment, and/or being configured for bone fixation in an acromioclavicular joint dislocation, wherein the first bone is the clavicle and the second bone is the coracoid process.

15. An arrangement, including:
a bone fixation device for fixation of a first bone and a second bone to each other in a bone fracture and/or a joint dislocation, including:
a first fixation element configured for passing through a hole of the first bone and a hole of the second bone and for engaging the second bone,
a flexible strand, and
a second fixation element configured for engaging the first bone and for securing the flexible strand,
wherein the first fixation element:
includes a hollow cylinder, wherein the hollow cylinder extends in a direction of a longitudinal axis from a first end to a second end and has a wall defining a cylinder hollow space and,
is configured for receiving the flexible strand; and,
a pusher for pushing the first fixation element of the bone fixation device through the hole of the first bone and the hole of the second bone,
wherein the pusher includes at least one mating element that is configured to mate with at least one mating element of the first fixation element and/or wherein the pusher has a pusher hollow space for passing through the flexible strand.

16. A bone fixation method for fixation of a first bone and a second bone to each other in a bone fracture, in particular a lesser trochanter fracture, by means of a bone fixation device including a first fixation element) configured for passing through a hole of the first bone and a hole of the second bone and for engaging the second bone, a flexible strand, and a second fixation element configured for securing the flexible strand and engaging the second bone, wherein the first fixation element is further configured for receiving the flexible strand, the bone fixation method including the steps of:
forming a hole in the first bone and a hole in the second bone,
passing the first fixation element, with the flexible strand received by the first fixation element, through the hole of the first bone and through the hole of the second bone so that the first fixation element exits the hole of the second bone,
engaging the second bone by means of the first fixation element and the first bone by means of the second fixation element, and
fixing the first bone and the second bone to each other by securing the flexible strand to the second fixation element.

17. The bone fixation method according to claim 16, wherein the first fixation element is a plate-shaped element with at least one through hole, in particular two through holes, through which the flexible strand is passed.

18. The bone fixation method according to claim 16, wherein the first fixation element includes a hollow cylinder, wherein the hollow cylinder has a wall defining a cylinder hollow space and extends in a direction of a longitudinal axis from a first end to a second end.

19. A bone fixation method for fixation of a first bone and a second bone to each other in a joint dislocation, in particular in an acromioclavicular joint dislocation, by means of a bone fixation device including a first fixation element configured for passing through a hole of the first bone and a hole of the second bone and for engaging the second bone, a flexible strand, and a second fixation element configured for securing the flexible strand and engaging the second bone, wherein the first fixation element is configured for receiving the flexible strand and includes a hollow cylinder, wherein the hollow cylinder extends in a direction of a longitudinal axis from a first end to a second end and has a wall defining a cylinder hollow space, the bone fixation method including the steps of:
forming a hole in the first bone and a hole in the second bone,
passing the first fixation element, with the flexible strand received by the first fixation element, through the hole of the first bone and through the hole of the second bone so that the first fixation element exits the hole of the second bone,
engaging the second bone by means of the first fixation element and the first bone by means of the second fixation element, and
fixing the first bone and the second bone to each other by securing the flexible strand to the second fixation element.

20. The bone fixation method according to claim 18, wherein the first fixation element includes at least one opening that is configured for receiving the flexible strand, wherein the flexible strand is passed through the at least one opening, wherein the at least one opening is in particular formed in and/or on the wall of the hollow cylinder.

21. The bone fixation method according to claim 20, wherein the at least one opening includes two wall holes formed in the wall of the hollow cylinder, wherein the wall holes extend in a direction that is perpendicular or oblique to an outer circumferential surface and/or an inner circumferential surface of the hollow cylinder and arranged in a row in a direction from the first end to the second end, in particular in a direction parallel to the longitudinal axis, and/or arranged symmetrically to a central transverse axis of the hollow cylinder, and wherein the wall holes are through holes and communicate with the cylinder hollow space.

22. The bone fixation method according to claim 18, wherein the first fixation element is passed through the hole of the first bone and the hole of the second bone by passing the first fixation element over a guiding element, in particular a guidewire, for guiding the first fixation element, the guiding element being inserted in the hole of the first bone and the hole of the second bone, in particular wherein the guiding element is removed after the first fixation element has exited the hole of the second bone.

23. The bone fixation method according to claim 16, wherein the step of passing the first fixation element through the hole of the first bone and the hole of the second bone includes pushing the first fixation element through the hole of the first bone and the hole of the second bone by means of a pusher, wherein the pusher preferably includes at least one mating element and the first fixation element includes at least one mating element being mated with the at least one mating element of the pusher for pushing the first fixation element.

24. The bone fixation method according to claim 23, wherein the first fixation element is pushed through the hole of the first bone and the hole of the second bone while keeping the flexible strand in tension, in particular wherein the flexible strand is kept in tension by being preloaded in a pusher hollow space.

25. The bone fixation method according to claim 24, wherein the tension is released when the first fixation element has exited the hole of the second bone, in particular by releasing the flexible strand from the pusher.

26. The bone fixation method according to claim 16, wherein the step of forming the hole of the first bone and the hole of the second bone includes the step of advancing a Kirschner wire, preferably a threaded Kirschner wire, through the first bone and the second bone.

27. The bone fixation method according to claim 26, wherein the step of forming the hole of the first bone and the hole of the second bone further includes the step of passing a cannulated drill over the Kirschner wire for drilling the hole of the first bone and the hole of the second bone.

28. The bone fixation method according to claim 27, wherein, while maintaining the cannulated drill in place, the Kirschner wire is removed after the hole of the first bone and the hole of the second bone have been formed, and a guidewire, in particular an atraumatic wire, for guiding the first fixation element is then inserted in the cannulated drill, and wherein the cannulated drill is then removed from the hole of the first bone and the hole of the second bone.

29. The bone fixation method according to claim 26, wherein the Kirschner wire is guided through a guide sleeve, which is directly placed against the proximal cortex of the first bone or inserted in a protective sleeve that is directly placed against the proximal cortex of the first bone, in particular wherein the guide sleeve is removed before passing the cannulated drill over the Kirschner wire.

30. The bone fixation method according to claim 16,
wherein, for engaging the second bone by means of the first fixation element, the flexible strand is pulled after the first fixation element has exited the hole of the second bone, and/or
wherein, for engaging the first bone by means of the second fixation element, the second fixation element is placed on the first bone or the flexible stand is pulled, wherein the second fixation element is loaded on the flexible strand such and/or the flexible strand is configured such that pulling the flexible strand when received by the first fixation element and the second fixation element causes the second fixation element to move in a direction opposite to the pulling direction for engaging the first bone.
